(19) United States
(12) Patent Application Publication
(10) Pub. No.: US 2005/0097632 A1
(43) Pub. Date: May 5, 2005

(54) Phenotype modifying genetic sequences

(76) Inventor: Bernard John Carroll, Moffat Beach (AU)

Correspondence Address: DUANE MORRIS LLP, 380 LEXINGTON AVENUE, NEW YORK, NY, 10168-0002, US

(21) Appl. No.: 10/923,286

(22) Filed: Aug. 20, 2004

Related U.S. Application Data
(63) Continuation of application No. 09/701,926, filed on Jun. 1, 2001, now abandoned.

(30) Foreign Application Priority Data
Jun. 4, 1998 (AU) PP 3901
Jun. 4, 1998 (AU) PP 3903
Sep. 25, 1998 (AU) PP 6169
Sep. 25, 1998 (AU) PP 6174

(51) Int. Cl. A01H 1/00; C12N 15/82; C12N 5/04

(52) U.S. Cl. 800/278; 800/284; 435/468; 435/419

(57) ABSTRACT

Nucleic acid molecules capable of modifying phenotypic traits in eukaryotic cells and in particular plant cells are described. The nucleic acid molecules of the present invention are referred to as “phenotype modifying genetic sequences” or “PMGSs” and may be used to increase and/or stabilise or otherwise facilitate expression of nucleotide sequences being expressed into a translation product. Alternatively, PMGSs may be used to down regulate by, for example, promoting transcript degradation via mechanisms such as co-suppression. In plant and non-plant cells, the PMGSs of the invention may also be used to inhibit, reduce or otherwise down regulate expression of a nucleotide sequence, such as a eukaryotic gene, including a pathogen gene, the expression of which results in an undesired phenotype.

The present invention relates generally to nucleic acid molecules capable of edifying phenotypic traits in eukaryotic cells and in particular plant cells. The nucleic acid molecules of the present invention are referred to as “phenotype modifying genetic sequences” or “PMGSs” and may be used to increase and/or stabilise or otherwise facilitate expression of nucleotide sequences being expressed into a translation product or may be used to down regulate by, for example, promoting transcript degradation via mechanisms such as co-suppression. The PMGSs of the present invention are also useful in modulating plant physiological processes such as but not limited to resistance to plant pathogens, senescence, cell growth, expansion and/or divsion and the shape of cells, tissues and organs. One particularly useful group of PMGSs modulate starch metabolism and/or cell growth or expansion or division. Another useful group of PMGSs are involved in increasing and/or stabilising or otherwise facilitating expression of nucleotide sequences in eukaryotic cells such as plant cells and in particular the expression of therapeutically, agriculturally and economically important transgenes. The PMGSs may also be used to inhibit, reduce or otherwise down regulate expression of a nucleotide sequence such as a eukaryotic gene, including a pathogen gene, the expression of which, results in an undesired phenotype. The PMGSs of the present invention generally result, therefore, in the acquisition of a phenotypic trait or loss of a phenotypic trait.

Bibliographic details of the publications numerically referred to in this specification are collected at the end of the description.

The subject specification contains nucleotide and amino acid sequence information prepared using the programme PatentIn Version 2.0, presented herein after the bibliography. Each nucleotide or amino acid sequence is identified in the sequence listing by the numeric indicator <210> followed by the sequence identifier (e.g. <210>1, <210>2, etc). The length, type of sequence (DNA, protein (PRT), etc) and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (eg. <400>1, <400>2, etc).

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Recombinant DNA technology is now an integral part of strategies to generate genetically modified eukaryotic cells. For example, genetic engineering has been used to develop varieties of plants with commercially useful traits and to produce mammal cells which express a therapeutically useful gene or to suppress expression of an unwanted gene. Transposons have played an important part in the genetic engineering of plant cells and some non-plant cells to provide inter alia tagged regions of genomes to facilitate the isolation of genes by recombinant DNA techniques as well as to identify important regions in plant genomes responsible for certain physiological processes.

The maize transposon Activator (Ac) and its derivative Dissociation (Ds) was one of the first transposon systems to be discovered (1, 2) and was used by Fedoroff et al (3) to clone genes. The behaviour of Ac in maize has been studied extensively and excision occurs in both somatic and germline tissue. Studies have highlighted two important features of Ac/Ds for tagging. First, the transposition frequency and second, the preference of Ac/Ds for transposition into linked sites.

The use of the Ac/Ds system has been hampered by the difficulty of data interpretation. One reason for this is the high activity of Ac in certain plants causing insertions at unlinked sites due to multiple transpositions, rather than a single event, from the T-DNA. This problem was addressed by Jones et at (4), Carroll et at (S) and others, and a two component Ac/Ds system was developed. In this system, Ds elements were made wherein the Ac transposase gene was replaced with a marker gene thereby rendering it non-autonomous. Separate Ac elements were then made. Subsequently, T-DNA regions of binary vectors carrying either a Ds element or a stabilised Activator transposase gene (sAc) were constructed by Carroll et at (5) and Scofield et al (6).

The Ds element contained a reporter gene (eg. nos:BAR) which was shown to be inactivated on crossing with plants carrying the sAc (5). This is referred to as transgene silencing. It has been shown that transgene silencing is a more general phenomenon in transgenic plants (7, 8, 9). Many different types of transgene silencing have now been reported in the literature and include: co-suppression of a transgene and a homologous endogenous plant gene (10), inactivation of ectopically located homologous transgenes in transgenic plants (7), the silencing of transgenes leading to resistance to virus infection (11) and inactivation of transgenes inserted in maize transposons in transgenic tomato (5).

Gene silencing undoubtedly reflects mechanisms of great importance in the understanding of plant gene regulation. It is of particular importance because it represents a severe obstacle to stable and high level expression of economically important transgenes (7).

In work leading up to the present invention, the inventors sought to identify regulatory mechanisms involved in controlling expression of phenotypic traits in eukaryotic cells and in particular plant cells including modulating plant physiological processes, preventing or otherwise reducing gene silencing and/or facilitating increased and/or stabilized gene expression in eukaryotic cells such as plant cells. In accordance with the present invention, the subject inventors have identified and isolated phenotype modifying genetic sequences referred to herein as “PMGSs” which are useful in modifying expression of nucleotide sequences in eukaryotic cells such as plant cells.

One aspect of the present invention is predicated in part on the elucidation of the molecular basis of transposase-mediated silencing of genetic material located within a transposable element. Although, in accordance with the present invention, the molecular basis of gene silencing has been determined with respect to plant selectable marker genes within the Ds element of the Ds/Ac maize transposon system, the present invention clearly extends to the silencing of any nucleotide sequence and in particular a transgene and to mechanisms for alleviating gene silencing. In accordance with the present invention, nucleotide sequences have been identified which alleviate gene silencing and which increase or stabilise expression of genetic material. Furthermore although the present invention is particularly exemplified in relation to plants, it extends to all eukaryotic cells such as cells from mammals, insects, yeasts, reptiles and birds.

Accordingly, an aspect of the present invention provides an isolated nucleic acid molecule comprising a sequence of nucleotides which increases or stabilizes expression of a second nucleotide sequence inserted proximal to said first mentioned nucleotide sequence.

The term “proximal” is used in its most general sense to include the position of the second nucleotide sequence near, close to or in the genetic vicinity of the first mentioned nucleotide sequence. More particularly, the term “proximal” is taken herein to mean that the second nucleotide sequence precedes, follows or is flanked by the first mentioned nucleotide sequence. Preferably, the second nucleotide sequence is within the first mentioned nucleotide sequence and, hence, is flanked by portions of the first nucleotide sequence. Generally, the second nucleotide sequence is flanked by up to about 10 kb either side of first mentioned nucleotide sequence, more preferably up to about 5 kb, even more preferably up to about 1 kb either side of said first mentioned nucleotide sequence and even more preferably up to about 10 bp to about 1 kb.

Another aspect of the present invention is directed to an isolated nucleic acid molecule comprising a sequence of nucleotides which stabilises, increases or enhances expression of a second nucleotide sequence inserted into, flanked by, adjacent to or otherwise proximal to the said first mentioned nucleotide sequence.

The second mentioned nucleotide sequence is preferably an exogenous nucleotide sequence meaning that it is either not normally indigenous to the genome of the recipient cell or has been isolated from a cell's genome and then re-introduced into cells of the same plant or animal, same species of plant or animal or a different plant or animal. More preferably, the exogenous sequence is a transgene or a derivative thereof which includes parts, portions, fragments and homologues of the gene.

The first mentioned nucleotide sequence described above is referred to herein as a “phenotype modulating genetic sequence” or “PMGS” since it functions to and is capable of increasing or stabilizing expression of an exogenous nucleotide sequence such as a transgene or its derivatives. This in turn may have the effect of alleviating silencing of an exogenous nucleotide sequence or may promote transcript degradation such as via co-suppression. The latter is particularly useful as a defense mechanism against pathogens such as but not limited to plant viruses and animal pathogens.

Accordingly, another aspect of the present invention relates to a PMGS comprising a sequence of nucleotides which increases, enhances or stabilizes expression of a second nucleotide sequence inserted within, adjacent to or otherwise proximal to said PMGS.

PMGSs may or may not be closely related at the nucleotide sequence level although they are closely functionally related in modulating phenotypic expression. Particularly preferred PMGSs are represented in<400>1; <400>2; <400>3; <400>4; <400>5; <400>6; <400>7; <400>8; <400>9; <400>10; <400>11; <400>12; <400>13; <400>14; <400>15; <400>16; <400>17; <400>18; <400>19; <400>20; <400>21; <400>22; <400>23; <400>24; <400>25; <400>26; <400>27; <400>28; <400>29; <400>30 and/or <400>31 as well as nucleotide sequences having at least about 25% similarity to any one of these sequences after optimal alignment with another sequence of a sequence capable of hybridizing to any one of these sequences under low stringency conditions at 42° C.

The term “similarity” as used herein includes exact identity between compared sequences at the nucleotide or amino acid level. Where there is non-identity at the nucleotide level, “similarity” includes difference between sequences which result in different amino acids that are nevertheless related to each other at the structural, functional, biochemical and/or conformational levels. Where there is non-identity at the amino acid level, “similarity” includes amino acids that are nevertheless related to each other at the structural functional, biochemical and/or conformational levels. In a particularly preferred embodiment, nucleotide and sequence comparisons are made at the level of identity rather than similarity. Any number of programs are available to compare nucleotide and amino acid sequences. Preferred programs have regard to an appropriate alignment. One such program is Gap which considers all possible alignment and gap positions and creates an alignment with the largest number of matched bases and the fewest gaps. Gap uses the alignment method of Needleman and Wunsch (24). Gap reads a scoring matrix that contains values for every possible GCG symbol match. GAP is available on ANGIS (Australian National Genomic Information Service) at website http://mell.angis.org.au. Another particularly useful programme is “tBLASTx” (25).

Reference herein to a low stringency at 42° C. includes and encompasses from at least about 0% v/v to at least about 15% v/v formamide and from at least about 1M to at least about 2M salt for hybridisation, and at least about 1M to at least about 2M salt for washing conditions. Alternative stringency conditions may be applied where necessary, such as medium stringency, which includes and encompasses from at least about 16% v/v to at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v formamide and from at least about 0.01M to at least about 0.15M salt for hybridisation, and at least about 001M to at least about 0.15M salt for washing conditions.

Accordingly, another aspect of the present invention provides a PMGS comprising the nucleotide sequence:
20<400>1; <400>2; <400>3; <400>4; <400>5; <400>6; <400>7; <400>8; <400>9; <400>10; <400>11; <400>12; <400>13; <400>14; <400>15; <400>16; <400>17; <400>18; <400>19; <400>20; <400>21; <400>22; <<400>26; <400>27; <400>28; <400>29; <400>30 and/or <400>31; or a sequence having at least 25% similarity after optimal alignment of said sequence to any one of the above sequences or a sequence capable of hybridizing to any one of the above sequences under low stringency conditions at 42° C.

Alternative percentage similarities or identities include at least about 30%, 40%, 50%, 60%, 70%, 80%, 90% or above.

A further aspect of the present invention is predicated on transposon-mediated tagging of tomato plants which was shown to result in the identification of mutants exhibiting altered physiological properties. In particular, the insertion of a transposon in close proximity to the α-amylase gene resulted in continued or modified expression of the α-amylase gene past the initial development stage of the plant. In wild-type plants, negative regulatory mechanisms which may include methylation result in the non-expression of the α-amylase gene. In accordance with this aspect of the present invention, modified expression of the α-amylase gene, post or after initial developmental stage, results in physiological attributes such as altered senescence, altered resistance to pathogens, modification of the shape of plant cells, tissues and organs and altered cell growth or expansion or division characteristics. It is proposed, in accordance with the present invention, that the altered physiological phenotype is due to modified starch metabolism by the continued or modified expression of the α-amylase gene. In particular, increased or modified expression of the α-amylase gene or otherwise continued or altered expression of the α-amylase gene post initial development results in cell death, i.e. cell apoptosis, but also induces or promotes resistance to pathogens.

Accordingly, another aspect of the present invention contemplates a method for controlling physiological processes in a plant said method comprising modulating starch metabolism in cells of said plant.

More particularly, the present invention is directed to a method of inducing a physiological response in a plant said method comprising inhibiting or facilitating starch metabolism in cells of said plant after the initial developmental stage.

This aspect of the present invention is exemplified herein with respect to the effects of starch metabolism in tomato plants. This is done, however, with the understanding that the present invention extends to the manipulation of starch metabolism in any plant such as flowering plants, crop plants, ornamental plants, vegetable plants, native Australian plants as well as Australian and non-Australian trees, shrubs and bushes. The preferred means of modulating physiological process is via the introduction of a PMGS. In this context, a nucleotide sequence encoding an α-amylase gene or a portion or derivative thereof or a complementary sequence thereto, for example, would be regarded as a PMGS, as would a nucleotide sequence which promotes increased and/or stabilised expression of a target gene.

The term “expression” is conveniently determined in terms of desired phenotype. Accordingly, the expression of a nucleotide sequence may be determined by a measurable phenotypic change involving transcription and translation into a proteinaceous product which in turn has a phenotypic effect or at least contributes to a phenotypic effect. Alternatively, expression may involve induction or promotion of transcript degradation such as during co-suppression resulting in inhibition, reduction or otherwise down-regulation of translatable product of a gene. In the latter case, the nucleic acid molecules of the present invention may result in production of sufficient transcript to induce or promote transcript degradation. This is particularly useful if a target endogenous gene is to be silenced or if the target sequence is from a pathogen such as a virus, bacterium, fungus or protozoan. In all instances “expression” is modulated but the result is conveniently measured as a phenotypic change resulting from increased or stabilised production of transcript thereby resulting in increased or stabilised translation product, or increased or enhanced transcript production resulting in transcript degradation leading to loss of translation product (such as in co-suppression).

The term “modulating” is used to emphasis that although transcription may be increased or stabilised, this may have the effect of either permitting stabilised or enhanced translation of a product or inducing transcription degradation such as via co-suppression.

Physiological responses and other phenotypic changes contemplated by the present invention include but are not limited to transgene expression, cell apoptosis, senescence, pathogen resistance, cell, tissue and organ shape and plant growth as well as cell growth, expansion and/or division.

In a particularly preferred embodiment, starch metabolism is stimulated, promoted or otherwise enhanced or inhibited by manipulating levels of an amylase and this in turn may lead to inter alia senescence or apoptosis as well as resistance to pathogens. Reference to “amylase” includes any amylase associated with starch metabolism including α-amylase and β-amylase. This aspect of the present invention also includes mutant amylases. In addition, the manipulation of levels of amylase may be by modulating endogenous levels of a target plants own amylase, or an exogenous amylase gene or antisense, co-suppression or ribozyme construct may be introduced into a plant. The exogenous amylase gene may be from another species or variety of plant or from the same species or variety or from the same plant. The present invention extends to recombinant amylases and derivative amylases including fusion molecules, hybrid molecules and amylases with altered substrate specifications and/or altered regulation. Any molecule capable of acting as above including encoding an α-amylase is encompassed by the term “PMGS”.

According to another aspect of the present invention there is provided a method of inducing a physiological response in a plant such as but not limited to inducing resistance to a plant pathogen, enhancing or delaying senescence, modifying cell growth or expansion or division or altering the shape of cells, tissues or organs, said method comprising modulating synthesis of an amylase or functional derivative thereof for a time and under conditions sufficient for starch metabolism to be modified.

Preferably, the amylase is α-amylase.

The manipulation of amylase levels may also be by manipulating the promoter for the amylase gene. Again, the introduction of a PMGS may achieve such manipulation. Alternatively, an exogenous amylase gene may be introduced or an exogenous promoter designed to enhance expression of the endogenous amylase gene. A PMGS extends to such exogenous amylase genes and promoters.

One group of PMGSs of the present invention were identified following transposon mutagenesis of plants with the Ds/Ac transposon system. The Ds element carries a reporter gene (nos:BAR) which is normally silenced upon exposure to the transposase gene. In a few cases, plants are detected in which nos:BAR expression is not silenced. In accordance with the present invention, it has been determined that the Ds element inserts within, adjacent to or otherwise proximal with a PMGS which results in increased or stabilized expression of the nos:BAR. In other words, the PMGS facilitates expression of a gene and preferably an exogenous gene or a transgene. This in turn may result in a gene product being produced or induction of transcript degradation such as via co-suppression.

The PMGSs of the present invention are conveniently provided in a genetic construct.

Accordingly, another aspect of the present invention contemplates a genetic construct comprising a PMGS as herein defined and means to facilitate insertion of a nucleotide sequence within, adjacent to or otherwise proximal with said PMGS.

The term “genetic construct” is used in its broadest sense to include any recombinant nucleic acid molecule and includes a vector, binary vector, recombinant virus and gene construct.

The means to facilitate insertion of a nucleotide sequence include but are not limited to one or more restriction endonuclease sites, homologous recombination, transposon insertion, random insertion and primer and site-directed insertion mutagenesis. Preferably, however, the means is one or more restriction endonuclease sites. In the case of the latter, the nucleic acid molecule is cleaved and another nucleotide sequence ligated into the cleaved nucleic acid molecule.

Preferably, the inserted nucleotide sequence is operably linked to a promoter in the genetic construct.

According to this embodiment, there is provided a genetic construct comprising an PMGS as herein defined and means to facilitate insertion of a nucleotide sequence within, adjacent to or otherwise proximal with said PMGS and operably linked to a promoter.

Conveniently, the genetic construct may include or comprise a transposable element such as but not limited to a modified form of a Ds element A modified form of a Ds element includes a Ds portion comprising a PMGS and a nucleotide sequence such as but not limited to a reporter gene, a gene conferring a particular trait on a plant cell or a plant regenerated from said cell or a gene which will promote co-suppression of an endogenous gene.

Another aspect of the present invention contemplates a method of increasing or stabilising expression of a nucleotide sequence or otherwise preventing or reducing silencing of a nucleotide sequence or promoting transcription degradation of an endogenous gene in a plant or animal or cells of a plant or animal, said method comprising introducing into said plant or animal or plant or animal cells said nucleotide sequence flanked by, adjacent to or otherwise proximal with a PMGS.

In an alternative embodiment, there is provided a method of inhibiting, reducing or otherwise down-regulating expression of a nucleotide sequence in a plant or animal or cells of a plant or animal said method comprising introducing into said plant or animal or plant or animal cells the nucleotide sequence flanked by, adjacent to or otherwise proximal with a PMGS.

Yet another aspect of the present invention provides a transgenic plant or animal carrying a nucleotide sequence flanked by, adjacent to or otherwise proximal to a PMGS. As a consequence of the PMGS, the expression of the exogenous nucleotide sequence is increased or stabilised resulting in expression of a phenotype or loss of a phenotype.

Although not intending to limit the present invention to any one theory or mode of action, one group of PMGSs is proposed to comprise a methylation resistance sequence. A methylation resistance sequence is one which may de-methylate and/or prevent or reduce methylation of a nucleotide sequence such as a target nucleotide sequence.

The present invention further extends to a transgenic plant or a genetically modified plant exhibiting one or more of the following characteristics:
(i) an amylase gene not developmentally silenced;
(ii) an amylase gene capable of constitutive or inducible expression;
(iii) a mutation preventing silencing of an amylase gene;
(iv) a nucleic acid molecule proximal to an amylase gene and which substantially prevents methylation of said amylase gene;
(v) decreased amylase gene expression; and/or
(vi) a genetically modified amylase allele(s).

Reference herein to a “gene” is to be taken in its broadest context and includes:
(i) a classical genomic gene consisting of transcriptional and/or translational regulatory sequences and/or a coding region and/or non-translated sequences (i.e. introns, 5′- and 3′-untranslated sequences)
(ii) mRNA or cDNA corresponding to the coding regions (i.e. exons) optionally comprising 5′- or 3′-untranslated sequences of the gene; or
(iii) an amplified DNA fragment or other recombinant nucleic acid molecule produced in vitro and comprising all or a part of the coding region and/or 5′- or 3′-untranslated sequences of the gene.

The term “proximal” is used in its most general sense to include the position of the amylase gene near, close to or in the genetic vicinity of the nucleic acid molecule referred to in part (iv) above. More particularly, the term “proximal” is taken herein to mean that the amylase gene precedes, follows or is flanked by the nucleic acid molecule. Preferably, the amylase is within the nucleic acid molecule and, hence, is flanked by portions of the nucleic acid molecule. Generally, the amylase gene is flanked by up to about 100 kb either side of the nucleic acid molecule, more preferably up to about 10 kb, even more preferably to about 1 kb either side of the nucleic acid molecule and even more preferably up to about 10 bp to about 1 kb.

Accordingly, another aspect of the present invention is directed to a PMGS comprising a sequence of nucleotides which stabilises, increases or enhances expression of an amylase gene inserted into, flanked by, adjacent to or otherwise proximal to the said nucleic acid molecule.

In an alternative embodiment, the present invention contemplates a PMGS comprising a sequence of nucleotides which inhibits, decreases or otherwise reduces expression of an amylase gene inserted into, flanked by, adjacent to or otherwise proximal to the said nucleic acid molecule.

The term “expression” is conveniently determined in terms of desired phenotype. Accordingly, the expression of a nucleotide sequence may be determined by a measurable phenotypic change such as resistance to a plant pathogen, enhanced or delayed senescence, altered cell growth or expansion or division or altered cell, tissue or organ shape.

The PMGS of this aspect of the present invention functions to and is capable of modulating expression of an amylase gene or its derivatives. The term “modulating” includes increasing or stabilising expression of the amylase gene or decreasing or inhibiting the amylase gene. The PMGS may be a co-suppression molecule, ribozyme, antisense molecule, an anti-methylation sequence, a methylation-inducing sequence and/or a negative regulatory sequence, amongst other molecules.

Accordingly, another aspect of the present invention relates to a PMGS comprising a sequence of nucleotides which increases, enhances or stabilizes expression of an amylase gene inserted within, adjacent to or otherwise proximal with said PMGS.

In an alternative embodiment, the present invention provides a PMGS comprising a sequence of nucleotides which inhibits, decreases or otherwise reduces expression of an amylase gene inserted within, adjacent to or otherwise proximal with said PMGS.

Another aspect of the present invention contemplates a genetic construct comprising a PMGS as herein defined and means to facilitate insertion of a nucleotide sequence within, adjacent to or otherwise proximal with said PMGS wherein said nucleotide sequence encodes an amylase or functional derivative thereof.

Preferably, the amylase gene sequence is operably linked to a promoter in the genetic construct.

According to this embodiment, there is provided a genetic construct comprising an PMGS as herein defined and means to facilitate insertion of a nucleotide sequence within, adjacent to or otherwise proximal with said PMGS and operably linked to a promoter wherein said nucleotide sequence encodes an amylase or functional derivative thereof.

Conveniently, the genetic construct may be a transposable element such as but not limited to a modified form of a Ds element. A modified form of a Ds element includes a Ds portion comprising a PMGS and a nucleotide sequence such as but not limited to a reporter gene and a gene encoding an amylase.

Another aspect of the present invention contemplates a method of increasing or stabilising expression of a nucleotide sequence encoding an amylase or otherwise preventing or reducing silencing of a nucleotide sequence encoding an amylase in a plant cell said method comprising introducing into said plant or plant cells said nucleotide sequence encoding an amylase flanked by, adjacent to or otherwise proximal with a PMGS.

In an alternative embodiment, the present invention provides a method of inhibiting, decreasing or otherwise reducing expression of a nucleotide sequence encoding an amylase in a plant cell said method comprising introducing into said plant or plant cells said nucleotide sequence encoding an amylase flanked by, adjacent to or otherwise proximal with a PMGS.

Yet another aspect of the present invention provides a transgenic plant carrying a nucleotide sequence encoding an amylase flanked by, adjacent to or otherwise proximal with a PMGS.

Still a further aspect of the present invention provides nucleic acid molecules encoding apoptotic peptides, polypeptides or proteins or nucleic acid molecules which themselves confer apoptosis. One example of an apoptotic nucleic acid molecule is a molecule capable of inducing or enhancing amylase synthesis. Other molecules are readily identified, for example, by a differential assay. In this example, nucleic acid sequences (e.g. DNA, cDNA, mRNA) are isolated from wild type plants and mutant plants which exhibit enhanced or modified amylase gene expression. The differential assay seeks to identify DNA or mRNA molecules in the mutant plant or wild type plant which are absent in the respective wild type plant or mutant plant. Such nucleic acid molecules are deemed putative apoptosis-inducing or apoptosis-inhibiting genetic sequences. These molecules may have utility in regulating beneficial physiological processes in plants.

Another aspect of the present invention contemplates a method for controlling physiological processes in a plant said method comprising modulating cell shape and/or expansion and/or division or growth in said plant.

More particularly, the present invention is directed to a method of inducing a physiological response in a plant said method comprising enhancing or facilitating the manipulation of cell shape and/or expansion or division or growth in said plant.

This aspect of the present invention is based on the detection of a Ds insertion in the Dem gene in plants. The Dem gene is highly expressed in shoot and root apices. The resulting mutation results in genetically-modified palisade tissue. Mutant lines exhibiting altered cell shape or expansion or division or growth are selected and, in turn, further lines exhibiting such beneficial characteristics as increased levels of photosynthetic activity are obtainable. The two basic processes which contribute to plant shape and form are cell division and cell expansion or growth. By somatically tagging Dem the inventors have demonstrated that Dem is required for expansion or division or growth of palisade and adaxial epidermal cells during leaf morphogenesis. Therefore, the primary role of the DEM protein in plant morphogenesis in general is in cell expansion or division or growth rather than the orientation or promotion of cell division.

Accordingly, another aspect of the present invention provides a method of inducing a physiological response in a plant such as but not limited to inducing resistance to a plant pathogen, enhancing or delaying senescence, modifying cell growth or expansion or division or altering the shape of cells, tissues Or organs, said method comprising modulating expression of the Dem gene.

Still yet another aspect of the present invention relates to a transgenic plant or a genetically modified plant exhibiting one or more of the following properties:
(i) a Dem gene not developmentally silenced;
(ii) a Dem gene capable of constitutive or inducible expression;
(iii) a mutation preventing silencing of the Dem gene;
(iv) a nucleic acid molecule proximal to the Dem gene and which substantially prevents methylation of said Dem gene or demethylates the Dem gene;
(v) decreased Dem gene expression; and/or
(vi) a genetically modified Dem allele(s).

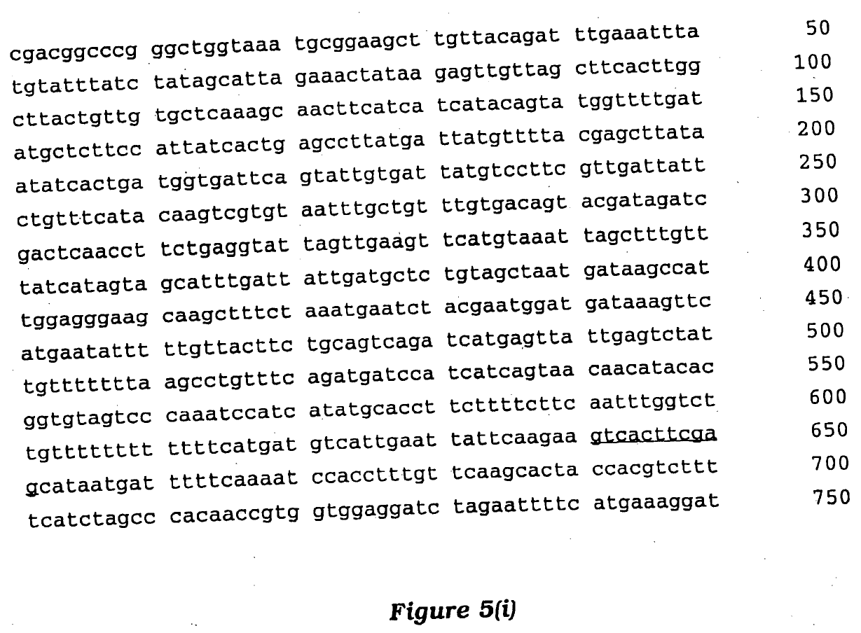

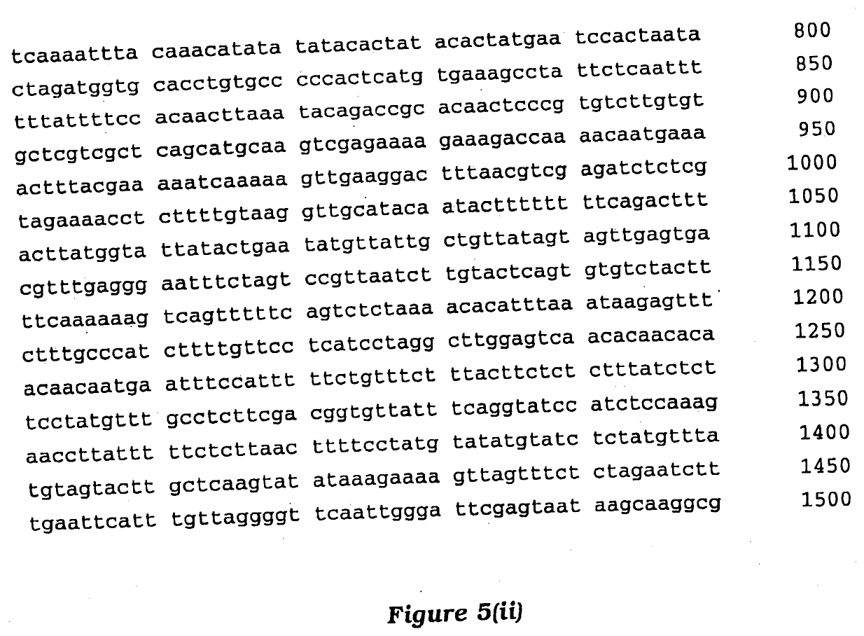

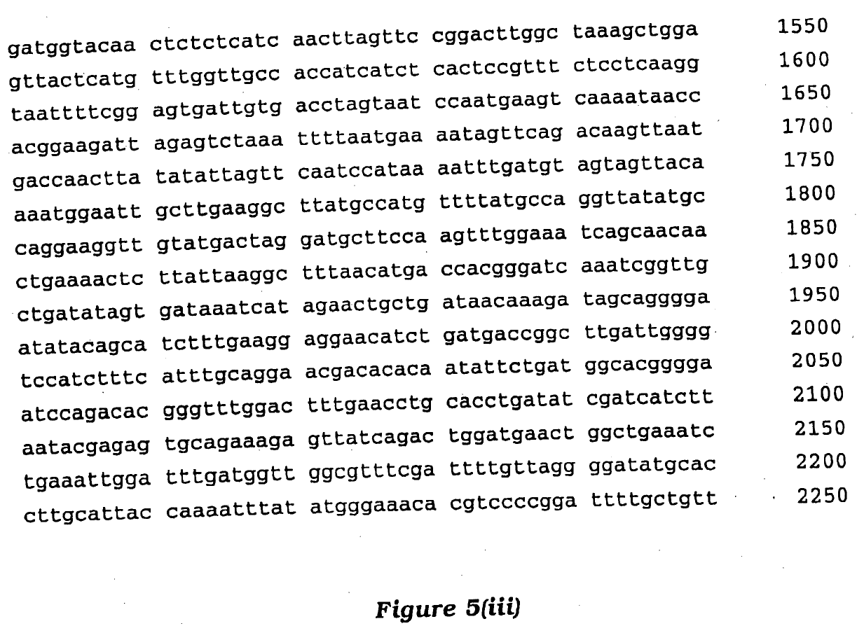

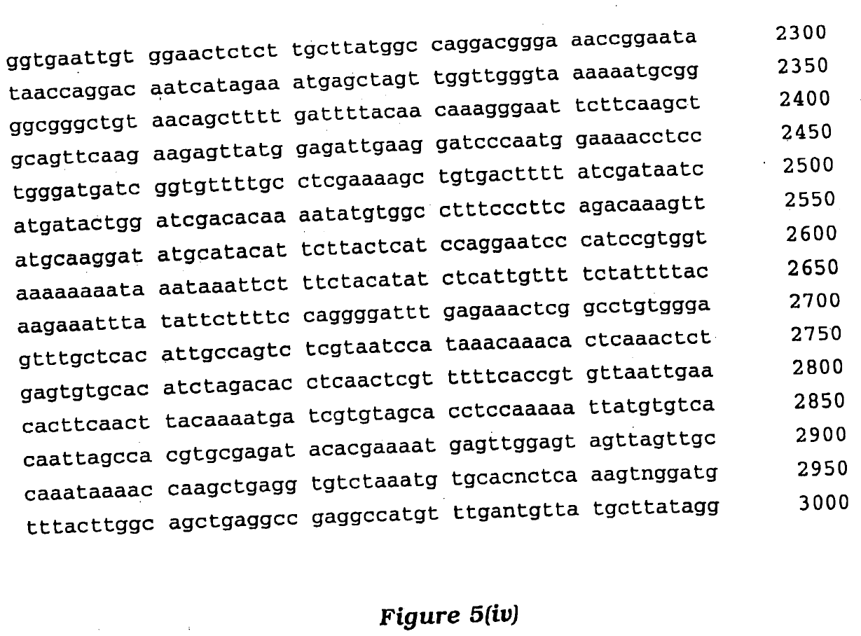

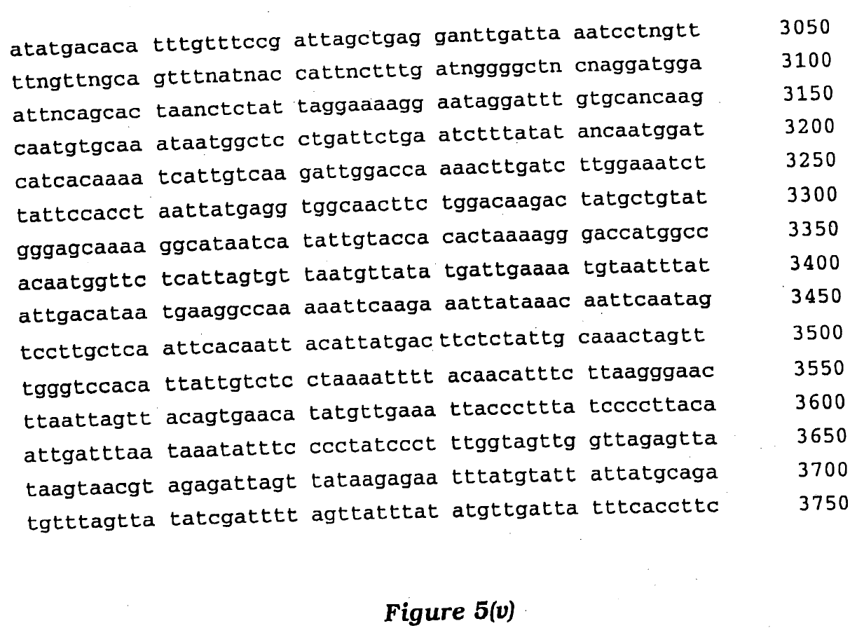

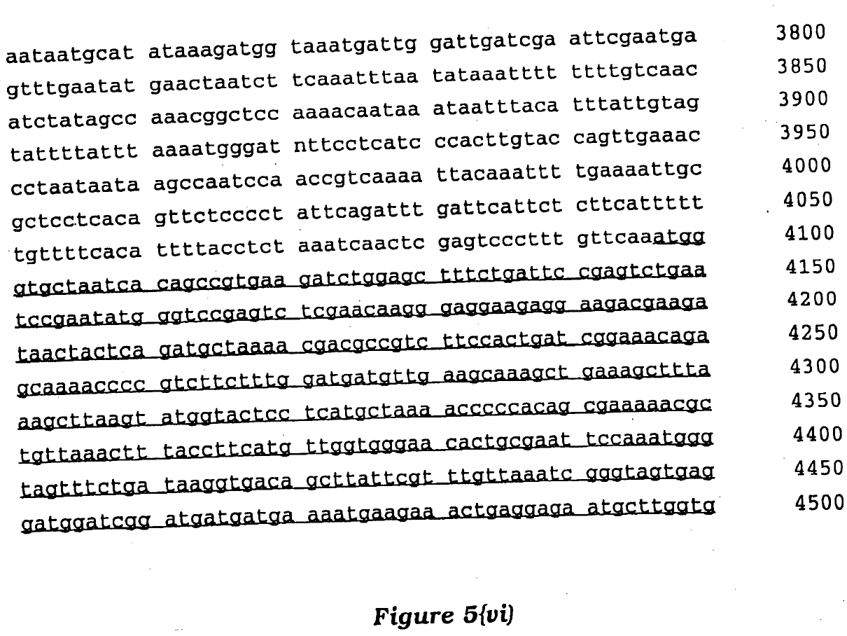

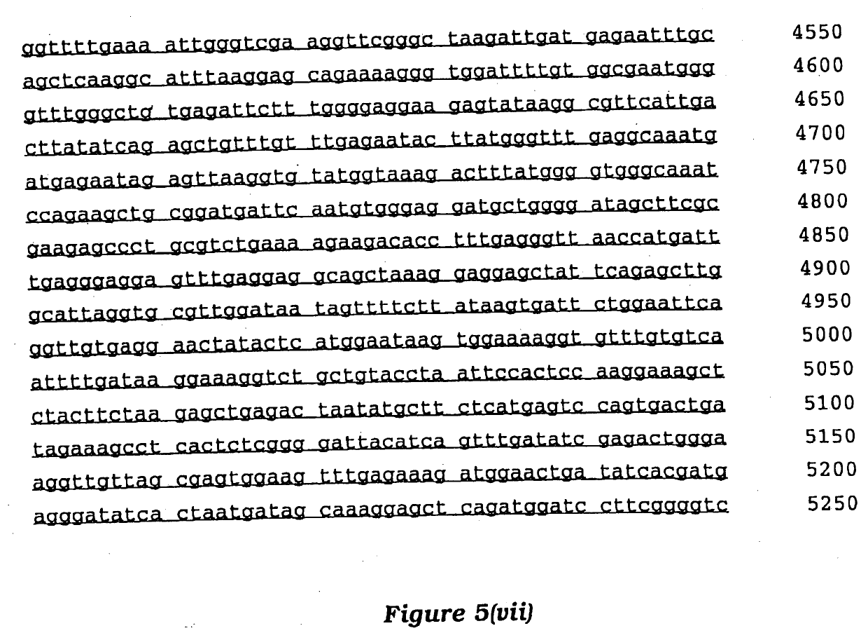

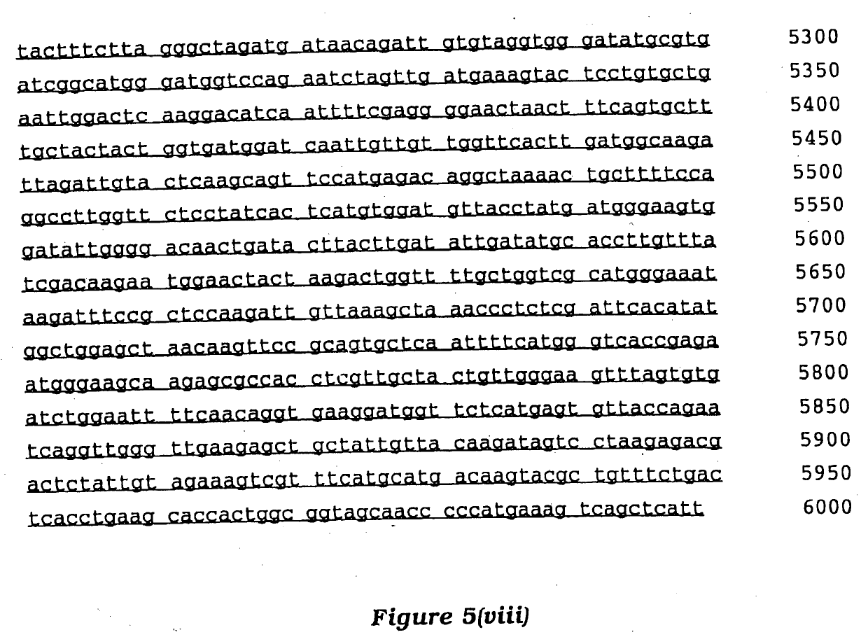

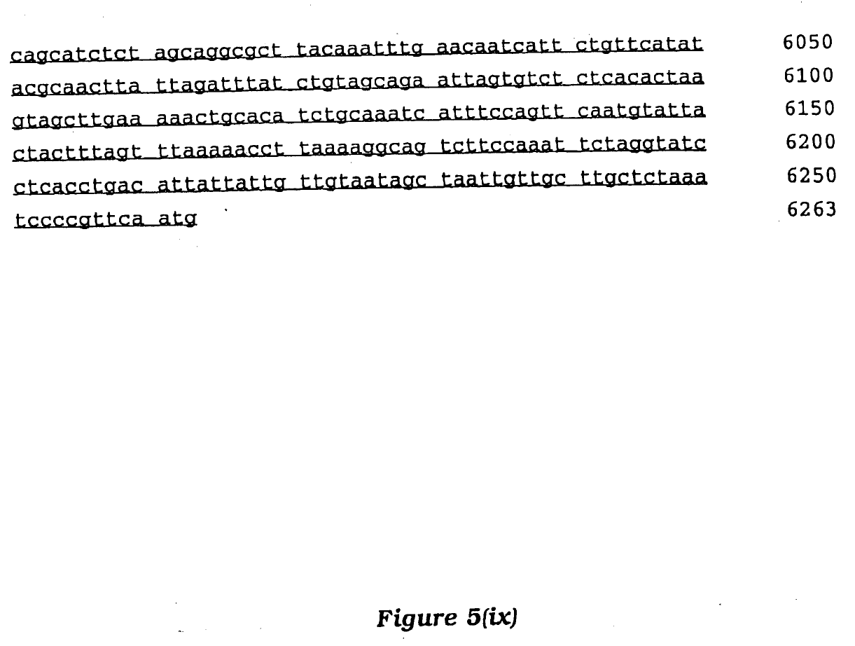

The present invention is further directed to the putative Dem promoter and its derivatives. The Dem promoter is approximately 700 bases in length extending upstream from the ATG start site. The nucleotide positions of putative Dem promoter are nucleotide 3388 to 4096 (FIG. 5). The nucleotide sequence of the Dem promoter is set forth in <400>8.

Yet another aspect of the present invention is directed to a mutation in or altered expression of a putative patatin gene in tomato or other plants. The patatin gene is referred to herein as “putative”, as it exhibits homology to the potato patatin gene.

Accordingly, another aspect of the present invention contemplates a method for controlling physiological processes in a plant said method comprising modulating C metabolism in cells of said plant.

More particularly, the present invention is directed to a method of inducing a physiological response in a plant said method comprising enhancing or facilitating C metabolism in cells of said plant.

Another aspect of the present invention provides a method of inducing a physiological response in a plant such as but not limited to inducing resistance to a plant pathogen, enhancing or delaying senescence, modifying cell growth or expansion or division or altering the shape of cells, tissues or organs, said method comprising modulating expression of a putative patatin gene or a functional derivative thereof.

Still yet another aspect of the present invention relates to a transgenic plant or a genetically modified plant exhibiting one or more of the following properties:
(i) a putative patatin gene not developmentally silenced;
(ii) a putative patatin gene capable of constitutive or inducible expression;
(iii) a mutation preventing silencing of a putative patatin gene;
(iv) a nucleic acid molecule proximal to a putative patatin gene and which substantially prevents methylation of said putative patatin gene or demethylates said putative patatin gene;
(v) decreased putative patatin gene expression; and/or
(vi) a genetically modified patatin allele(s).

Reference herein to “genetically modified” genes such as an altered amylase, Dem or patatin allele includes reference to altered plant development genes. The present invention is particularly directed to alteration of alleles which leads to economically physiologically or agriculturally beneficial traits.

The present invention further provides for an improved transposon tagging system.

One system employs a modified Ds element which now carries a PMGS.

Accordingly, another aspect of the present invention is directed to an improved transposon tagging system, said system comprising a transposable element carrying a nucleotide sequence flanked by, adjacent to or otherwise proximal with a PMGS.

Another new system employs the Dem gene or its derivatives as an excision marker. Reference to “derivatives” includes reference to mutants, parts, fragments and homologues of Dem including functional equivalents. The Dem gene is required for cotyledon development and shoot and root meristem function. Stable Ds insertion mutants of Dem germinate but fail to develop any further. However, unstable mutants in the Dem locus result in excision of the Ds element and reversion of the Dem locus to wild-type, thereby restoring function to the shoot meristem. In accordance with the present invention, the new system enables selection for transposition.

In accordance with the improved method, transposition is initiated by crossing a Ds-containing ime with a stabilized Ac (sAc)-containing line. The Ds-containing line is heterozygous for a Ds insertion in the Dem gene and the sAc line is heterozygous for a stable mutation in the Dem gene. A particularly useful mutant in the Dem gene is a stable mutation. Both of the Ds- and sAc-containing plant lines are wild-type due to the recessive nature of the Ds insertion and mutant alleles. The F₁ progeny derived from crossing the Dr and sAc lines segregate at a ratio of 3 wild-types to 1 mutant Because the sAc is linked to the frameshift dem allele, almost all of the F₁ mutants also inherit the transposase gene and can undergo somatic reversion. These revertant individuals have abnormal cotyledons, but Ds excision from the Dem gene restores function to the shoot apical meristem. Each somatic revertant represents an independent transposition event from the Dem locus. By screening for expression of a gene resident on the Ds element (e.g. nos:BAR), the identification of PMGSs is readily determined.

The present invention also provides in vivo bioassays for expressed transgenes. The bioassays identify nucleotide sequences which prevent transgene silencing.

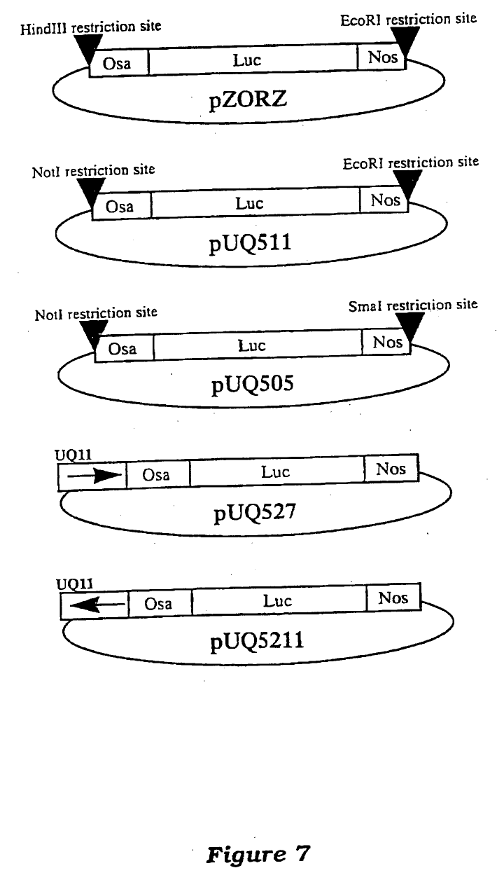

In one aspect, the plant expression vector pZorz carries a firefly luciferase reporter gene (luc), under the control of the Osa promoter (12). After bombardment, the gene is expressed in embryogenic sugarcane callus. However, it becomes completely silenced upon plant regeneration. The silencing appears to be correlated with methylation of the transgene. Genetic sequences flanking reactivated nos:BAR insertions are inserted into modified forms of the pZorz expression vector. These pZorz constructs are then used with a transformation marker to transform sugarcane in order to test whether the plant sequences are capable of alleviating silencing of the luc gene upon plant regeneration. Restriction endonuclease fragments capable of alleviating silencing of the luc gene are subject to deletion analysis and smaller fragments are subcloned into modified pZorz expression vectors to define the sequences more accurately (FIG. 7).

In another aspect, a plant expression vector is constructed for testing the PMGSs in Agrobacterium-transformed Arabidopsis. PMGSs are placed upstream of the nos:Luc or nos:Gus gene linked to a transformation marker and used to test whether PMGS s stabilise expression of the nos:Luc or nos:Gus gene in Arabidopsis.

These aspects of the present invention are clearly extendable to assays using other plants and the present invention contemplates the subject assay and plant expression vector for use in a range of plants in addition to sugar cane.

The present invention is further described by the following non-limiting Figures and Examples.

In the Figures:

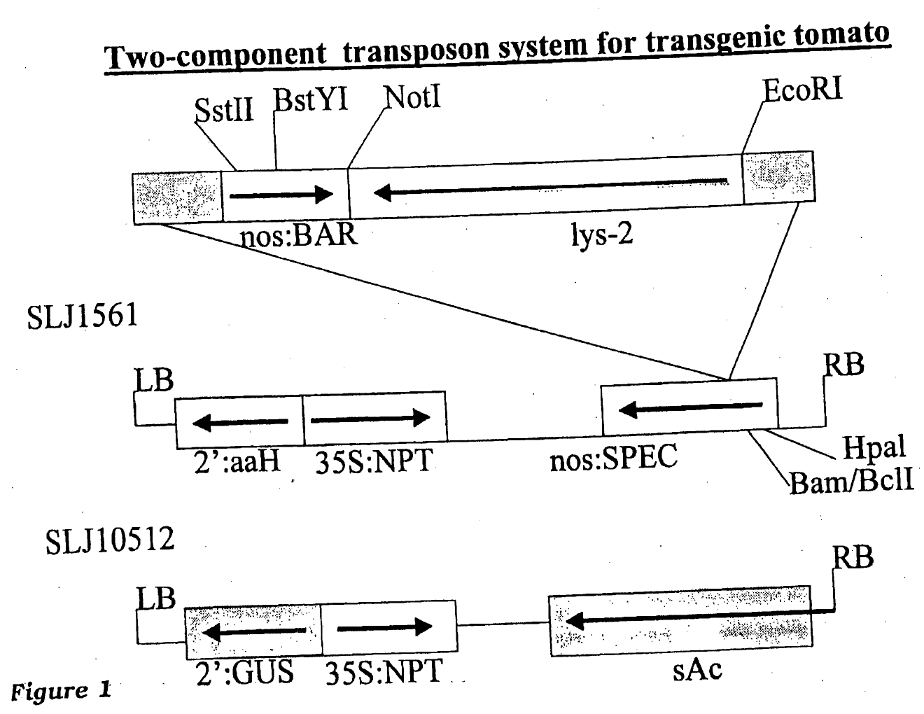

FIG. 1 is a diagrammatic representation showing T-DNA regions of binary vectors carrying a Ds element (SLJ1561) of the transposable gene (SLJ10512)[5]. The Ds element carries a nos:BAR gene and is inserted into a nos:SPEC excision marker. The transposon gene sAc is linked to a 2′:Gus reporter gene.

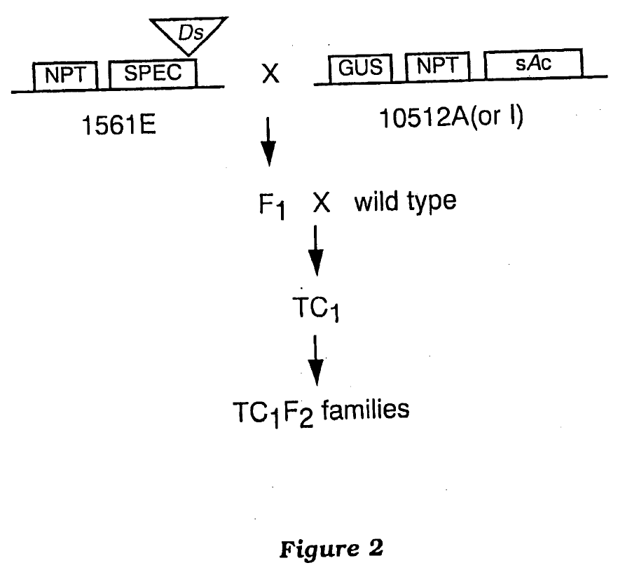

FIG. 2 is a diagrammatic representation showing an experimental strategy for generating tomato lines carrying transposed Ds elements (5). F1 plants heterozygous for both the Ds and sAc T-DNAs are test-crossed to produce TC, progeny. The TC₁ progeny are then screened for lines carrying a transposed Ds and a reactivated nos:BAR gene.

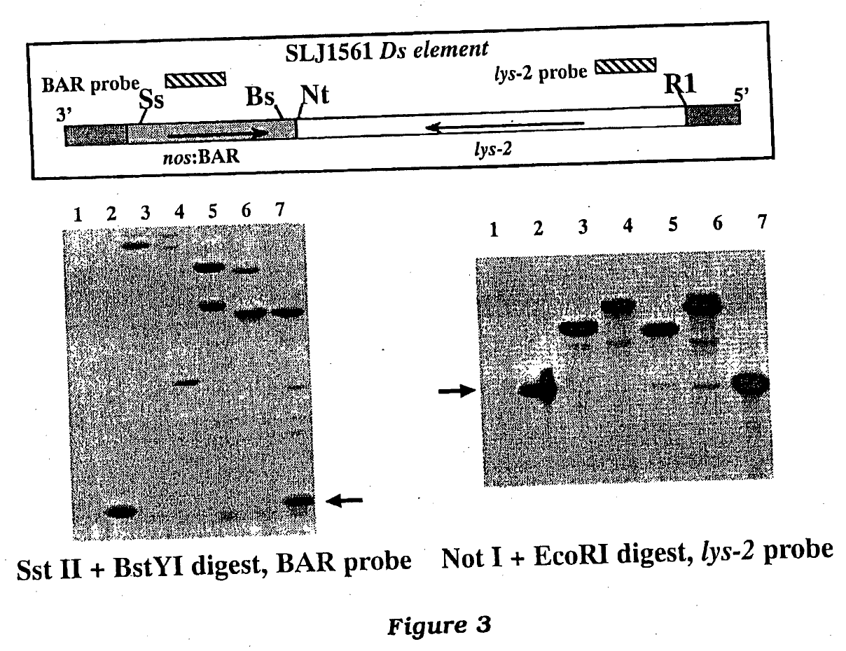

FIG. 3 is a representation showing methylation of a genetically engineered Ds transposon in transgenic tomato. Two separate Southern analyses were conducted on 7 individual genotypes; genomic DNA was extracted from leaf tissue (5). The restriction enzymes and probes (shaded boxes) used are shown on the figure. Lanes: 1. Non transformed (i.e. no Ds or nos:BAR gene), 2. 1561E which carries an active nos:BAR gene (due to the fact that it has never been exposed to the transposase gene), 3-6. Four tomato lines that carry silent nos:BAR genes, 7. UQ406 which carries an active nos:BAR gene due to insertion of the Ds in the α-amylase promoter. The enzymes SstII (abbreviated Ss) and NotI (abbreviated Nt) are methylation sensitive, whereas BstYI (abbreviated Bs) and EcoRI (abbreviated RI) are not. The expected size fragment for unmethylated DNA is indicated by the arrow; larger fragments (as in the silent lines) indicate methylation of the DNA at the SstII or NotI sites.

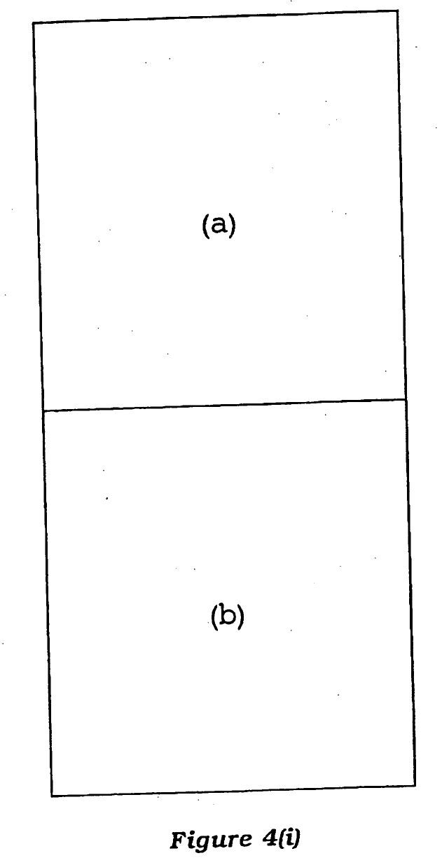

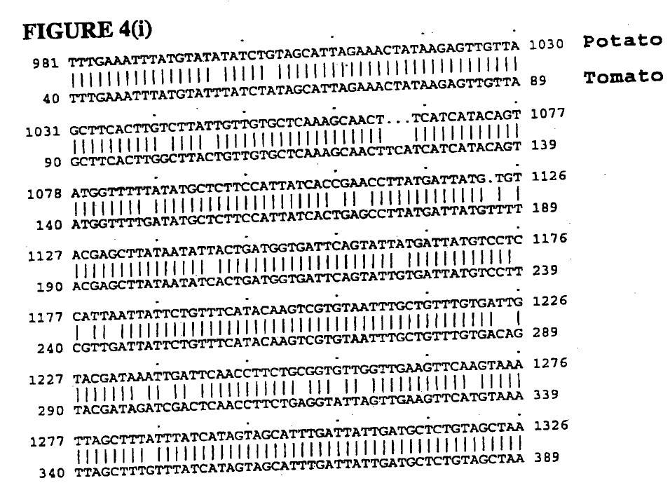

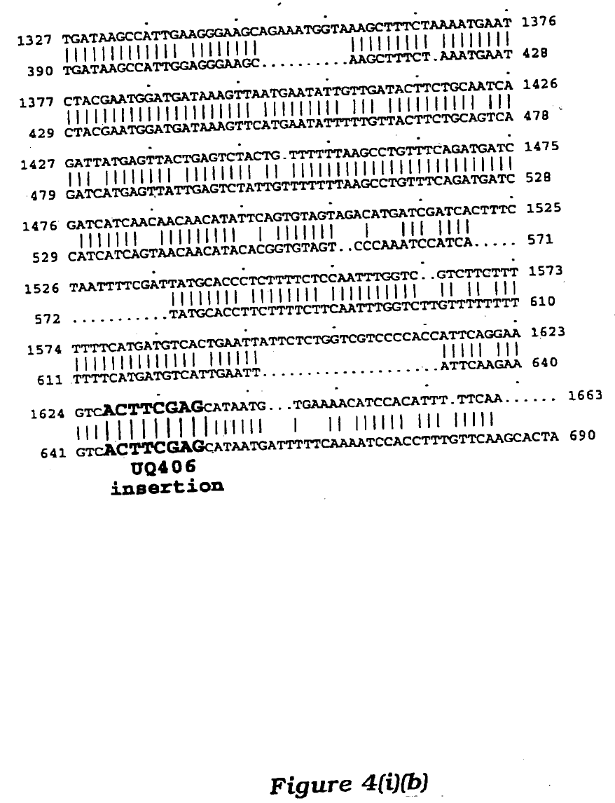

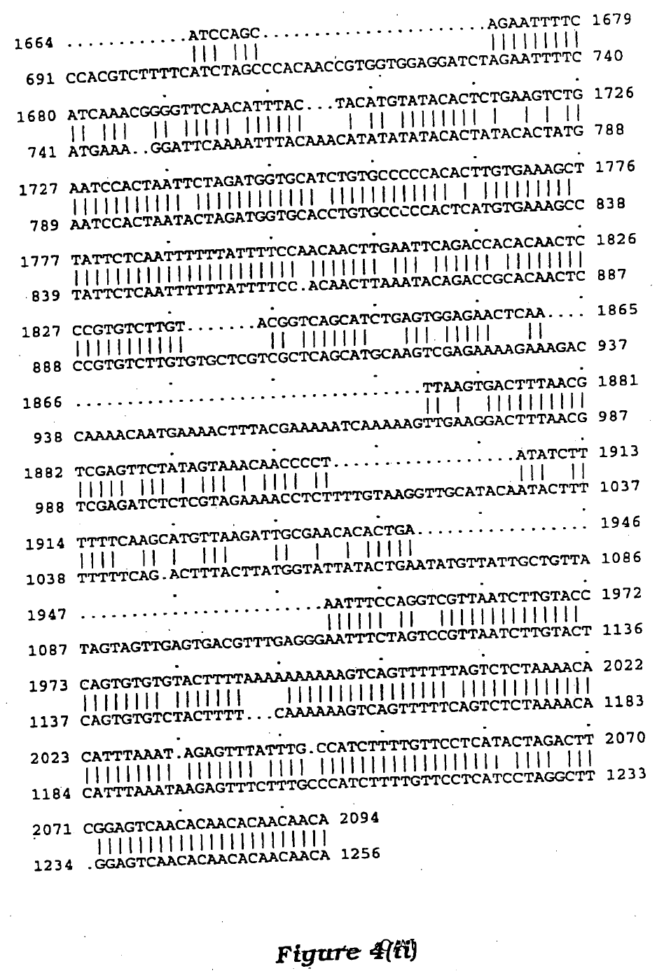

FIG. 4 is a representation showing a sequence comparison between the potato α-amylase promoter (15)<400>2 and the tomato amylase promoter <400>1. The location of the UQ406 insertion is shown.

FIG. 5 is a representation of a nucleotide sequence <400>3 of tomato genomic DNA from 651 bp upstream of the Ds insertion (acttcgag: underlined) in UQ406 to the beginning of the Dem coding sequence, followed by the Dem cDNA sequence from the ATG start site at base pair 4097 (sequence underlined). The target sequences of the Ds insertion in UQ406 and Dem ATG are underlined. The Dem cDNA sequence is shown in italics and underlined. The putative Dem promoter begins at nucleotide 3388 and ends just immediately prior to the ATG, i.e. at position 4096<400>8.

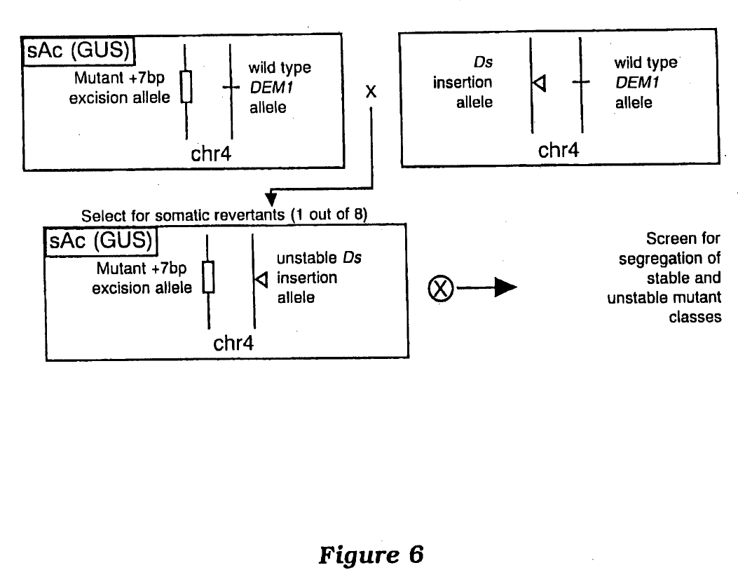

FIG. 6 is a diagrammatic representation showing an improved transposon tagging strategy using Dem as excision marker. The sAc and Ds parent lines are represented by the upper left and right boxes, respectively. Because the sAc is linked to the dem mutant +7 allele, somatic revertants can theoretically occur at about the frequency of 1 out of 4 in the F1 progeny. Each somatic revertant represents an independent transposition event. Chr4, chromosome 4 of tomato.

FIG. 7 is a diagrammatic representation showing construction of pUQ expression vectors from the pZorz vector (12; see Example 9).

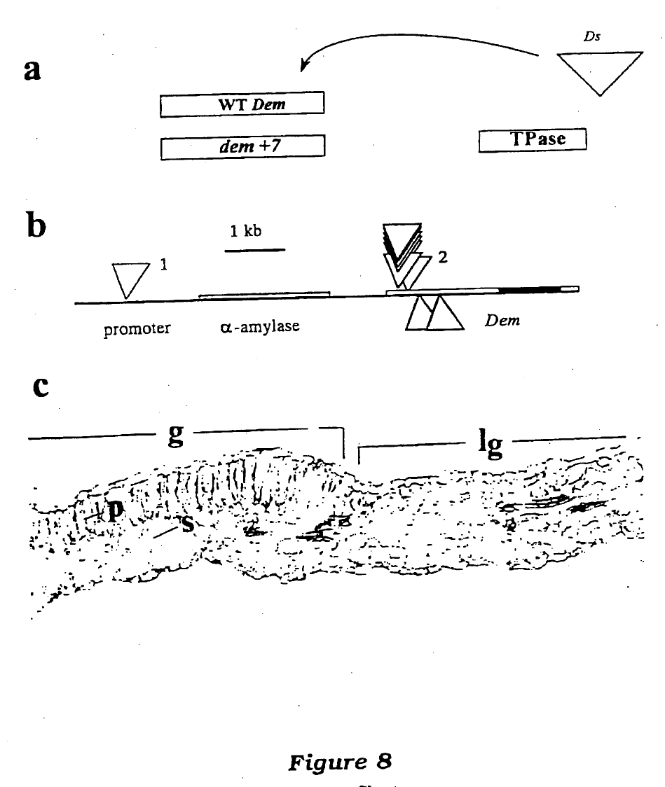

FIG. 8 is a representation of somatic tagging of the Dem locus. a. Diagrammatic representation of the STD (somatic tagging of Dem) genotype. dem+7 is a stable frameshift mutant of Dem, TPase represents a T-DNA 3 centiMorgans (cM) from Dem, carrying the Ac transposase and a GUS reporter gene. The transposase is required for Ds transposition. b. Location of stably inherited (shaded) and somatic (open) Ds insertions in the Dem locus and an upstream α-amylase gene. The α-amylase gene is in the same orientation as Dem. Coding sequences plus introns are shown as boxes and the dark section of the Dem locus represents an intron. All of the 8 somatic insertions shown in the figure were associated with palisade deficient sectors. The genomic region represented in b has been sequenced (see FIG. 5; please note that the intron in the dem locus is not included in this sequence). c. Mutant dem sectors lack palisade cells (p, palisade cells, s, spongy mesophyll, g, wild-type dark green sectors, and 1 g, mutant light green sectors).

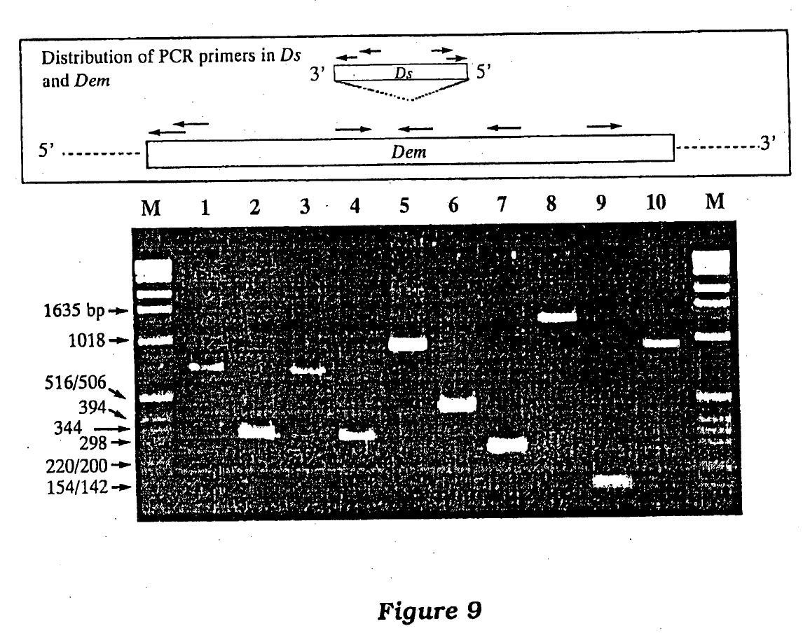

FIG. 9 shows PCR on intact tissue of dem sectors. M, 1 kb ladder. 1-10, unique Ds insertions in Dem detected by PCR. Intact leaf tissues (mutant somatic sectors) were used as template in the PCR. PCR with oligonucleotide primers facing out of Ds and in the Dem coding sequence amplified unique fragments from each mutant sector, thereby confirming that the sectors shown in FIG. 8 are indeed mutant dem sectors.

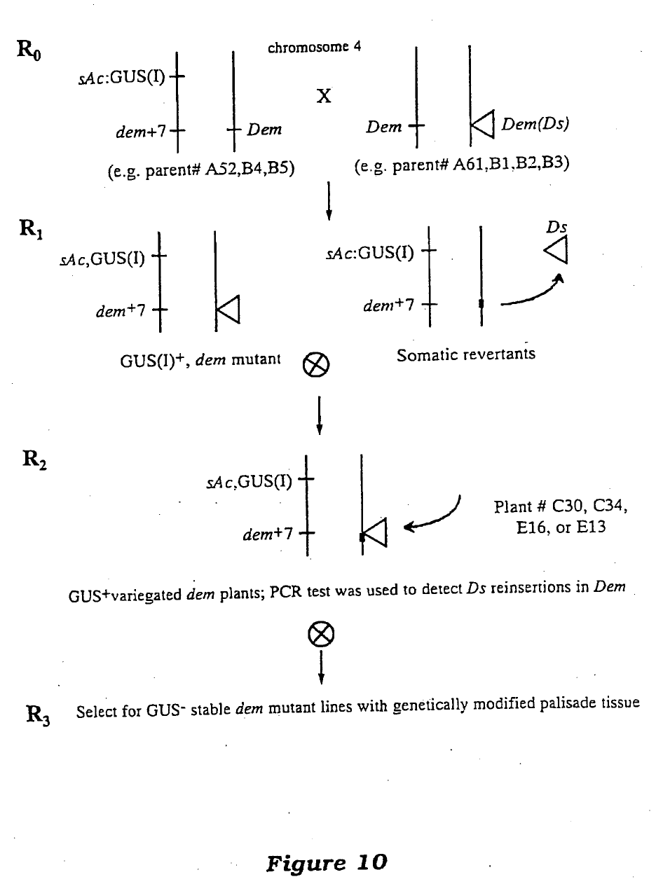

FIG. 10 is a diagrammatic representation of the genetic derivation of plants containing independent somatic dem alleles. Somatic revertants were generated by crossing plants heterozygous for the dem⁺⁷ mutant allele linked to transposase (sAc,GUS) and plants heterozygous for the dem^{Ds} mutant allele. Revertant seedlings were selfed and GUS⁺ individuals were identified. From 150 somatic revertants, four independent lines were produced carrying hundreds of independent dem alleles.

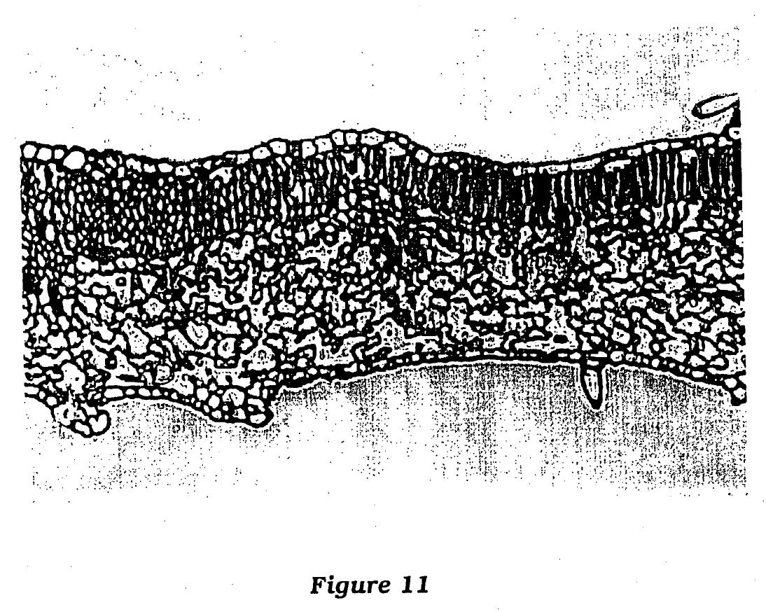

FIG. 11 is a photographic representation showing a multicellular palisade mutant allele of the Dem locus. At the single-cell embryo stage, the plant possessing the multicellular palisade sector shown carried a transposase gene and was heterozygous for a mutant frameshift allele and a wild-type allele of the Dem locus. During development, however, mutant dem sectors were produced due to the insertion of a Ds element into the wild-type allele. Wild-type palisade tissue is essentially composed of single long columnar cells. Some mutant sectors (due to Ds insertion) totally lack palisade cells (refer to the figure), whereas other mutant sectors have multicellular palisade tissue composed of small, non-columnar cells.

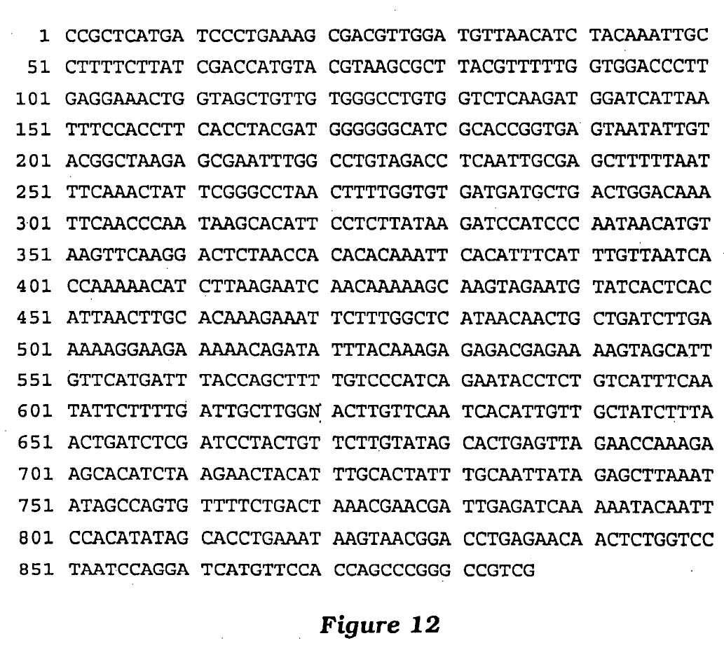

FIG. 12 is a representation of the nucleotide sequence upstream of the UQ11 Ds insertion. The UQ11 Ds insertion resulted from transposition of the Ds back into the T-DNA. Nucleotide is the first nucleotide upstream of Ds (containing an active nos:BAR gene). Nucleotide 1 to 295 correspond to Agrobacterium sequence form the right border of tomato transformant 1561E (5), the starting position of the Ds before loding in the Dem locus. Nucleotides 296 to 886 (in italics) correspond to tomato genomic DNA flanking the T-DNA insertion in 1561E. Note the BamHI/BcII fusion sequence (TGACTC) and the HpaI site (GTTAAC), both underlined in the figure immediately upstream of the insertion site. The putative PMGSs of UQ11 reside in the right border of the T-DNA (nucletoide 1 to 295), and/or the flanking tomato DNA (nucleotide 296 to 886), or further upstream.

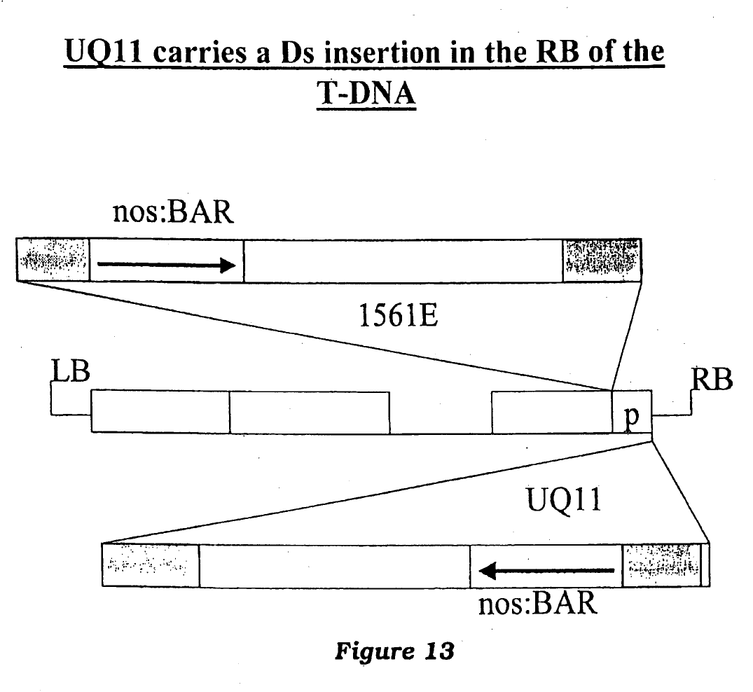

FIG. 13 is a diagrammatic representation of the T-DNA construct SLJ 1561 used to transform tomato to produce 1561E(5), and the location of the Ds element in UQ11. The Ds element in UQ11 is slightly closer to the right border (RB) and in the opposite orientation compared to the Ds element in 1561E.

TABLE 1
SUMMARY OF SEQUENCE (SEQ) IDENTIFIERS

| SEQ | |
|---|---|
| IDENTIFIER | DESCRIPTION |
| <400>1 | Nucleotide sequence of tomato α-amylase gene promoter |
| <400>2 | Nucleotide sequence of potato α-amylase gene promoter |
| <400>3 | Nucleotide sequence of genomic DNA upstream of Dem |
| | gene followed by Dem cDNA coding sequence in tomato |
| | line UQ406 |
| <400>4 | Nucleotide sequence upstream of Ds insertion (ie. |
| | upstream of the nos: BAR gene) in a putative patatin |
| | gene in tomato line UQ12 |
| <400>5 | Nucleotide sequence downstream of Ds insertion (ie. |
| | downstream of the nos: BAR gene) in a putative patatin |
| | gene in tomato line UQ12 |
| <400>6 | Nucleotide sequence of portion of putative tomato |
| | (UQ12) homologue of potato patatin gene |
| <400>7 | Nucleotide sequence of portion of potato patatin gene |
| | having homology to <400>6 |
| <400>8 | Nucleotide sequence of putative Dem promoter in |
| | UQ406 |
| <400>9 | Nucleotide sequence upstream of Ds insertion in tomato |
| | mutant UQ11 |
| <400>10 | Putative PMGS from UQ11 corresponding to nucleotides |
| | 1 to 295 of <400>9 |
| <400>11 | Putative PMGS from UQ11 corresponding to nucleotide |
| | 296 to 836 of <400>9 |
| <400>12 | Nucleotide sequence of an upstream portion of putative |
| | sucrose synthase gene in tomato (UQ14) containing |
| | PMGS |
| <400>13 | Nucleotide sequence of an downstream portion of |
| | putative sucrose synthase gene in tomato (UQ14) |
| | containing PMGS |
| <400>14 | Putative PMGS from UQ14 |
| <400>15 | Partial nucleotide sequence of 3′ untranslated region |
| | from potato sucrose synthase |
| <400>16 | PMGS from UQ14 |
| <400>17 | Partial nucleotide sequence of 3′ untranslated region |
| | from potato sucrose synthase |
| <400>18 | PMGS from UQ14 |
| <400>19 | Partial nucleotide sequence of 3′ untranslated region |
| | from potato lactate dehydrogenase (LDH) |
| <400>20 | PMGS from UQ14 |
| <400>21 | Partial nucleotide sequence of intron II of tomato |
| | phytochrome B1 (PHYB1) |
| <400>22 | PMGS from UQ14 |
| <400>23 | Partial nucleotide sequence of 3′ untranslated region |
| | from potato sucrose synthase |
| <400>24 | PMGS from UQ14 |
| <400>25 | Partial nucleotide sequence of 3′ untranslated region of |
| | potato lactate dehydrogenase (LDH) |
| <400>26 | PMGS from UQ14 |
| <400>27 | Partial nucleotide sequence of intron I of potato cytosolic |
| | pyruvate kinase (CPK) |
| <400>28 | PMGS from UQ14 |
| <400>29 | Partial nucletoide sequence downstream of Brassica |
| | napus 1.7S seed storage protein, napin (napA) |
| <400>30 | PMGS from UQ14 |
| <400>31 | Partial nucleotide sequence of 3′ untranslated region of |
| | tomato chorismate synthase 2 precursor gene (CSP) |
| <400>32 | Nucleotide sequence of an upstream portion of Ds insert |
| | containing PMGS in tomato (line UQ13) |
| <400>33 | Nucleotide sequence of an downstream portion of Ds |
| | insert containing PMGS in tomato (line UQ13) |
| <400>34 | PMGS from UQ13 |
| <400>35 | Partial nucleotide sequence of tomato expansin 2 |
| <400>36 | PMGS from UQ13 |
| <400>37 | Partial nucleotide sequence of tomato ADP-glucose |
| | pyrophosphorylase |
| <400>38 | PMGS from UQ12 |
| <400>39 | Partial nucleotide sequence of tomato Ca²⁺ ATPase |

### EXAMPLE 1

### Ds/sAc Transposon System

The inventors have previously developed a two component Ds/sAc transposon system in transgenic tomato for tagging and cloning important genes from plants (5, 13). The components of the system are shown in FIG. 1 and comprise: i) a non-autonomous genetically-engineered Ds element (e.g. SLJ1561), and ii) an unlinked transposase gene sAc (SLJ10512), required for transposition of the Ds element. To activate transposition, the two components are combined by crossing transformants for each component. A plant selectable marker gene, e.g. nos:BAR, is inserted into the Ds element to enable selection for reinsertion of the elements following excision from the T-DNA (FIG. 1). The marker gene is irreversibly inactivated when the Ds line is crossed to a transformant expressing the transposase gene (5). Silencing occurred when the Ds element remained in its original position in the T-DNA, and also occurred in the great majority of cases when the Ds element transposed to a new location in the tomato genome. The silenced marker gene has been shown to be stably inherited even after the transposase gene segregates away from the Ds element in subsequent generations.

### EXAMPLE 2

### Transposon Tagging of a Chromosomal Region Enabling Full Expression of the nos:BAR Transgene

The experimental strategy for generating tomato lines carrying transposed Ds elements from T-DNA 1561E is shown in FIG. 2. The Ds element in 1561E carries a nos:BAR marker gene. In construction of the Ds, the 5′ end of the nos promoter is cloned into the Xho I site, 1100 bp from the 3′ end of Ac. Hundreds of plants carrying transposed Ds elements are screened for resistance to phosphinothricin (PPT), the selection agent for the BAR gene. Surprisingly, several lines are identified which show at least some level of resistance. One line, called UQ406, carries a single transposed Ds element (without the transposase gene which has segregated away) and is resistant to PPT. Stable inheritance of BAR gene expression in this line has been demonstrated through several generations. These results indicate that the strategy for tagging active chromosomal regions by screening for PPT resistance is a successful approach. Southern hybridization analysis of the original Ds transformant 1561E, UQ406 and several lines carrying silenced nos:BAR transgenes indicates that silencing is correlated with methylation of the SstII site in the nos promoter (FIG. 3). Total leaf tissue is used in this analysis, and the SstII site in the nos promoter in UQ406 is only partially methylated, enabling sufficient expression of the bar gene to confer resistance. In silent nos:BAR genes, the SstII site and NotI site immediately downstream from the coding sequence are both methylated (FIG. 3). In UQ406, the NotI site is unmethylated, as in 1561E FIG. 3).

### EXAMPLE 3

### Cloning Sequences Flanking an Active nos:BAR Gene

GenomeWalker (14) is used to clone the tomato DNA sequences flanking the Ds element in UQ406. The DNA flanking the Ds element in line UQ406 is cloned and sequenced, and a search of the PROSITE database reveals that the Ds has inserted into the promoter region of an α-amylase gene. The promoter <400>1 shows strong similarity to an α-amylase promoter of potato (15; FIG. 4) <400>2 and the coding sequence of the gene has strong homology with one of 3 reported potato α-amylase cDNAs (16). The DNA from 651 bp upstream of the UQ406 insertion to the end of the Dem coding sequence, has been sequenced (FIG. 5) <400>3. Other such sequences have been located and cloned (see below) using the method of Example 4. Nucleotide sequences disclosed herein which flank the active nos:BAR gene are designated “phenotype modulating genetic sequences” or “PMGSs”.

### EXAMPLE 4

### An Improved Transposon Tagging Strategy for Transgenic Tomato

The inventors have used the transposon tagging system described in Example 1 (also see FIG. 2) to tag and clone two important genes involved in shoot morphogenesis. The DCL gene is required for chloroplast development and palisade cell morphogenesis (13) and the Dem (Defective Embryo and Meri stem) gene is required for cotyledon development and shoot and root meristem function. Stable Ds insertion mutants of Dem germinate but fail to develop any further. In contrast, the unstable Dem seedlings appear at first to be mutant but the transposase gene activates transposition of the Ds and reversion of the Dem locus to wild-type, thereby restoring function to the shoot meristem.

While the transposon tagging system described in FIG. 2 has been successful in tagging genes and a chromosomal region alleviating transgene silencing, it does have two associated inefficiencies. First, transposition cannot be selected in the shoot meristem of F₁ plants heterozygous for Ds and sAc. As a consequence, many TC, progeny derived from test-crossing these F₁ plants still have the Ds located in the T-DNA. The other limitation of the system is that sibling TC₁ progeny derived from a single F₁ plant often carry the same clonal transposition and reinsertion event. The extent of clonal events amongst sibling TC₁ progeny can only be monitored by time consuming and expensive Southern hybridisation analysis.

These two inefficiencies in the transposon tagging strategy are overcome in accordance with the present invention by using the Dem gene as an excision marker. The new system enables selection for transposition in the shoot apical meristem and visual identification of plants carrying independent transposition events. Transposition is initiated by crossing a Ds line with a sAc line (FIG. 6). The Ds line is heterozygous for a Ds insertion in the Dem gene and the sAc line is heterozygous for a stable frameshift mutation in the Dem gene (FIG. 6). The frameshift allele is derived from a Ds excision event from the Dem locus. Both the Ds and sAc lines are wild-type due to the recessive nature of the Ds insertion and frameshift alleles. PCR tests on intact leaf tissue have been developed for the rapid identification of these Ds and sAc parental lines. The F₁ progeny derived from crossing the Ds and sAc lines segregate at the expected ratio of 3 wild-types to 1 mutant. Because the sAc is linked to the frameshift dem allele, almost all of the F₁ mutants also inherit the transposase gene (sAc) and can undergo somatic reversion. These revertant individuals have abnormal cotyledons, but Ds excision from the Dem gene restores function to the shoot apical meristem. Each somatic revertant represents an independent transposition event from the Dem locus. A non-destructive test for nos:BAR expression is used involving application of phosphinothricine [PPT] (the selective agent for expression of BAR gene) to a small area of a leaf. Somatic revertants resistant to PPT are grown though to seed and the F₂ progeny are screened again for PPT resistance. Lines carrying transposed Ds elements expressing nos:BAR are selected for more detailed molecular analysis. Four additional independent insertions carry active nos:BAR genes. These mutants are UQ11, UQ12, UQ13 and UQ14. The donor Ds was originally located in the Dem gene (FIG. 3) and in that location in the Dem gene the nos:BAR gene was silent. These independent lines were selected for further analysis (see Examples 5 and 6).

The efficient saturation mutagenesis of this chromosomal region is dependent on the use of the Dem gene as a selectable marker for independent transposition events. A recombinant selectable marker for independent transpositions is produced and transformed into tomato for saturation mutagenesis in other chromosomal regions of tomato. This system may be introduced into any species possessing the dem mutation, in order to facilitate transposon tagging of genes.

### EXAMPLE 5

### Ds Transposon Tagging of a Putative Patatin Gene

DNA sequences flanking the active nos:BAR in a line designated UQ12 have similarly been cloned and sequenced. The flanking DNA appears to correspond to an intron in a homologous potato patatin gene. Patatin is the major protein in the potato tuber and has many potentially-important characteristics. For example, it possesses antioxidant activity; it has esterase activity and is potentially a phospholipase or lipid acylhydrolase (hydrolyzing phospholipase, liberating free fatty acids); it is induced during disease resistance; and it inhibits insect larval growth.

The sequence upstream of the Ds insertion (i.e. upstream of the nos:BAR gene) is as follows:

| | | | | | |
|---|---|---|---|---|---|
| AATCAAAGAG | GAATTNAATT | CCNCAAAATT | TCATCCATAG | ATTTTGNGTC | 50 |
| TCTGAAAATT | AAAGTGACTT | TGTAATCTGA | AACCTAGAGT | CCTCAACCAT | 100 |
| ATCATTGACC | ATTAAGCCAT | ACCCTTAAAT | GTAGGGAATT | TGAAGTTTTA | 150 |
| AAAACCACAC | TTTGTTATTT | ATTGGCCCAA | ATACTCGATA | ATCTTTACAT | 200 |
| TATTGAAAAT | CAACATTCAA | AAGGAACGAA | CCTTCAATCA | CACCATCAAT | 250 |
| GTCAACTTTC | TTTTATTTTG | GATAATCTAA | GTTTTTAAAT | TGCAGTAAAA | 300 |
| TNAAATAAAA | CCCTAAACTT | CTTCTAGGTT | GAGACTTAGT | AAATATGAAT | 350 |
| TATATAAAGA | ATTCATGACA | AATGAGACAT | AAGAATAGTG | CCAGCAAATT | 400 |
| ACTTTTTTGA | TATCTTATCT | GTGATATCGG | AATTTTAACT | ACCATAAATT | 450 |
| TATGAATGAA | ATATCACTTA | TCTATTAGAG | AGGATTTAAT | CTCCCTTATA | 500 |
| ATGACATTGA | TAAAAGCAAG | NACAAGTGCT | CTTTATTTCT | TAATTACAAA | 550 |
| TCCTTAAATA | GATAAAAGCT | ACGAATAACA | TAATATCCTT | AAATAGATAA | 600 |
| AAGCTACGAA | TAACATAATA | GTATATTACT | CCNAATTATT | TTGATTTATT | 650 |

| | | | | |
|---|---|---|---|---|
| TAAAATGACT | CCACTAATCC | TGATGTGGTC | TAGG <400>4 | 684 |

The tomato sequence immediately downstream of the Ds insertion (i.e. downstream of the nos:BAR gene) is as follows:

| | | | | | |
|---|---|---|---|---|---|
| GGTCTAGGCC | CTGGGTCTAG | GAAACAAAAT | AACTTATTTG | ACTCCTAAAC | 50 |
| AATAGCAACA | TACAAACCAC | TGATATTGTA | CAAGTAAAAT | TCAATAAAAT | 100 |
| TCTAGCTCTC | TCAAACACTT | TTAAAATTGT | TATTTCTGTT | TTGTCTGTGT | 150 |
| CATATTATGA | CCTACACAAC | AACAACAACA | ACGAATTTAG | TGAAACTCTA | 200 |
| CAAAGTGGAG | CCTGAAGTCG | AGAGTTTACG | CGGGCCTTAT | CACTATCTTT | 250 |
| TCGAGATAAA | AAAATTATTT | TTAAAAGATC | ATCGACTTAA | ACAAACCAAA | 300 |
| CAATAATTAA | AAAAATATGA | ATTAATAGCA | AAGCAGTGTG | GACCATATAT | 350 |
| ACAAAAATCT | ATAACAACAA | CAAGGTGCAG | AGCATTATTC | CAACTAAGAT | 400 |
| CGAAGTTGTG | ATACTGTCAT | AATAAAAATG | ACACATATTT | TGACAACATA | 450 |
| AAAAATAAAT | AACCATAAAA | TATATCATAG | AAAAATGAAT | ATATTAGAAC | 500 |
| AGCTCACTCC | AATATTAAAA | GAGAGAAAAA | AAATATTTTC | CCACCACAAT | 550 |
| GCCATAATCC | TTGAGCTTAG | CTATTTATAA | GTAAAAAAAA | TGTTTTCTTG | 600 |
| GATAAATAGA | AAAAGAAATA | ATAATTAAAC | ATAACCAATC | ACTTCACAAA | 650 |

| | | |
|---|---|---|
| TAAGAGTGTA | TT <400>5 | 662 |

The level of homology between the potato and a tomato sequence is as follows:

| | | | |
|---|---|---|---|
| Tomato: | 307 | ATTTATTTTTAGGAAAAATTATCTAAATACACATCTTATTTTACCATATACTCTAAAAAT | 246 |
| | | \| \|\|\|\| \|\|\|\|\|\|\|\|\|\|\|\|\|\| \|\|\|\|\|\|\|\|\|\| \|\|\|\| \| \|\| \|\|\|\| \|\|\|\|\|\|\|\| \| | |
| Potato: | 1914 | AATTATATTTAGGAAAAATTACATAAATACACAACTTAATATATTATATTCTCTAAAATT | 1973 |

| | | | |
|---|---|---|---|
| 247 | TCC | 245 | <400>6 |
| | \|\|\| | | |
| 1974 | TCC | 1976 | <400>7 |

This Ds line also exhibits a disease mimic phenotype (as does UQ406), indicating that the patatin gene may be involved in disease resistance and/or may act as an antioxidant in plant cells.

Homology is determined between UQ12 and a partial sequence encoding Ca2+ ATPase:

Bestfit of UQ12D73 and Ca2+ ATPase

. . . . .

| | | |
|---|---|---|
| 914 | TTATACATTTCTGTTTGTATAAAGTGAAAGAGGAGAAGCAGAGAGTGGCG | 865 |
| | \|\|\|\|\| \|\|\|\| \| \|\|\|\|\|\|\|\|\|\|\|\|\|\|\|\|\|\|\| \|\| \| \|\|\|\|\|\| \|\|\| | |
| 1015 | TTATATATTTGTATTTGTATAAAGTGAAAGAGACGATG..GAGAGTAGCG | 1062 |

. . . . .

| | | |
|---|---|---|
| 864 | AGCGAGTTCCAGGAAGAGAAAAGAATGTCAATATGTTTTCTACGGATTAG | 815 |
| | \|\|\|\|\|\| \| \| \|\|\|\|\| \|\|\| \| \| \| \| \| \|\|\|\|\| | |
| 1063 | AGCGAGATTAAAAAAGAGTGGCGAACG.....AGATATGCCGTAAATTAG | 1107 |

. . . . .

| | | |
|---|---|---|
| 814 | AATTAAATGAAACTGTAGCTATATTATTTATTTTTAAATTAATAATTTGC | 765 |
| | \|\|\|\|\|\|\|\|\|\|\|\|\|\|\|\| \|\|\|\| \|\|\|\|\|\|\|\|\| \|\| \| \| \|\|\|\| | |
| 1108 | AATTAAATGAAACTGTCATTATAACATTTATTTTGAATAAATAATTTTGA | 1157 |

. . . .

| | | |
|---|---|---|
| 764 | TATAATGCACAAATTTCCTTTAAAACGAAAAAAGTATTTGATAATGT | 718 |
| | \|\|\|\|\|\| \|\|\|\|\| \|\|\|\| \|\|\|\|\|\| \| \|\| \| \|\|\|\|\|\|\|\| | |
| 1158 | TATAATACACAATTTTC..TTAAAAAGCAACGA......GATAATGT | 1196 |

### EXAMPLE 6

UQ11 Mutant Tomato Plant

A mutant tomato plant designed UQ11, was subject to characterization. The UQ11 Ds insertion resulted from transposition of the Ds back into the T-DNA, but it is slightly closer to the right border and in the opposite orientation (FIG. 13). FIG. 12 shows the DNA sequence upstream of the UQ11 Ds insertion. Nucleotide 1 is the first nucleotide upstream of the Ds (and the active nos:BAR gene). The sequence for nucleotides 1 to 295 is T-DNA sequence corresponding to the tight border of tomato transformant 1561E (5), the starting position of the Ds before lodging in the Dem locus. This is nucleotide sequence <400>10. Nucleotides 296 to 886 (in italics) [<400>11] correspond to tomato genomic DNA flanking the T-DNA insertion in 1561 E. Note the BamHI/BcII fusion sequence (TGATCC) and the HpaI site (GTTAAC), both in bold in the FIG. 12, immediately upstream of the insertion site (see FIG. 1). The putative PMGSs of UQ11 reside in the right border of the T-DNA (nucleotide 1 to 295), and/or the flanking tomato DNA (nucleotide 296 to 886). Another PMGS may also be located further upstream.

### EXAMPLE 7

### PMGS in Tomato Mutant UQ14

A Ds insertion mutant, UQ14, resulted in nos:BAR expression. The transposon had, therefore, inserted proximal to a PMGS. The nucleotide sequences comprising PMGSs are represented in 400>12 and <400>13.

A series of comparisons between <400>12 and other genes or nucleotide sequences was conducted:

(1) Homology between PMGS-UQ14 sequence [<400>14] upstream of Ds insertion and the 3′ untranslated region of a potato sucrose synthase (susi) gene, Acc. no. AP067860 (70% homologous over about 200 bp):

| | | . . . . . | |
|---|---|---|---|
| PMGS-UQ14 | 40 | TATGTTGCTCAAATCCTTCAAAAATCTCGACAGATGCATG.........G | 80 |
| | | \|\|\|\|\|\|\|\|\|\|\|\|\| \|\|\|\|\|\|\|\|\|\| \|\| \|\|\|\| \|\|\| \|\| | |
| Potato susi | 7549 | TATGTTGCTCAAACACTTCAAAAATGTCCACAGGTGCGTGTCGGATACTC | 7598 |
| | | . . . . . | |
| PMGS-UQ14 | 81 | CACCCGGTAGTGCATTTTTTTGAATGAGCTGGATACGAGTGCAATAATAT | 130 |
| | | \|\| \|\|\|\|\|\| \|\|\|\| \|\| \|\| \|\|\|\| \|\|\| \| \|\|\| | |
| Potato susi | 7599 | CAAAAAGTAGTGTATTTAGGTGTGTG....TGATATTAGT...AGTGTAT | 7641 |
| | | . . . . . | |
| PMGS-UQ14 | 131 | ATTTGGGAAGTTTGAGCAAAATAGACCTGAAATTACTTTTAGCTTTTCTT | 180 |
| | | \|\|\|\| \|\| \|\| \|\| \| \| \| \|\|\| \|\| \| \|\|\| \| \| \| \| \|\| | |
| Potato susi | 7642 | ATTTAGG.TGTGTGTGGATAGTAG...TGTATTTAGATGTGTGTGATATT | 7687 |
| | | . . . . . | |
| PMGS-UQ14 | 181 | TTTTAAAG..............GAATCGGATATGGGTACAATAATATTTT | 216 |
| | | \| \|\|\|\| \|\|\|\| \|\|\|\| \|\|\|\| \| \|\| \| \|\|\| | |
| Potato susi | 7688 | TCAAAAAGTTGTGTATTTTGGAGAATTTGATACGGGTGCGGCAACAATTT | 7737 |
| | | . . | |
| PMGS-UQ14 | 217 | TGAAGAGTC.TGAGCAACATAG | 237 |
| | | \|\|\|\|\|\|\|\|\| \|\|\|\|\|\| \|\|\|\| | |
| Potato susi | 7738 | TGAAGAGTCAGGAGCAAAATAG | 7759 |

(2) Homology between Region 1 of PMGS-UQ14 sequence (upstream of Ds insertion) and 3′ untranslated regions of potato sucrose synthase and two other genes, namely:
   a) 3′ untranslated region of a potato sucrose synthase (susi) gene, Acc. no. AF067860 (83% homologous over 41 bp),
   b) 3′ untranslated region of a potato lactate dehydrogenase (LDH) gene (85% homologous over about 41 bp), and

c) intron 2 of the tomato phytochrome B1 (PHYB1) gene, Acc. no LEAJ2281 (95% homologous over 22 bp).

| a) | | | |
|---|---|---|---|
| PMGS-UQ14 | 40 | TATGTTGCTCAAATCCTTCAAAAATCTCGACAGATGCATGGC | 81 |
| | | \|\|\|\|\|\|\|\|\|\|\|\|\| \|\|\|\|\|\|\|\|\|\| \|\| \|\|\|\| \|\|\| \|\| \| | |
| Potato susi | 7549 | TATGTTGCTCAAACACTTCAAAAATGTCCACAGGTGCGTGTC | 7590 |
| b) | | | |
| PMGS-UQ14 | 39 | CTATGTTGCTCAAATCCTTCAAAAATCTCGACAGATGCATG | 79 |
| | | \|\|\|\|\|\|\|\|\|\|\|\|\|\|\|\|\|\|\|\|\|\|\|\|\|\| \|\| \|\|\|\|\| \|\| | |
| Potato LDH | 704 | CTATGTTGCTCAAATCCTTCAAAAATGTCATTGGATGCGTG | 744 |
| c) | | | |
| PMGS-UQ14 | 40 | atgttgctcaaatccttcaaaaa | 62 |
| | | \|\|\|\|\|\|\|\|\|\|\|\|\|\|\|\| \|\|\|\|\|\| | |
| Tomato PHYB1 | 6781 | atgttgctcaaatcctccaaaaa | 6803 |

(3) Homology between Region 2 of PMGS-UQ14 sequence (upstream of Ds insertion) and untranslated regions of five other genes, namely:
   a) 3′ untranslated region of a potato sucrose synthase (susi) gene, Acc. no. AF067860 (74% homologous over 38 bp),
   b) 3′ untranslated region of a potato lactate dehydrogenase (LDH) gene (75% homologous over about 47 bp),
   c) intron 1 of a potato cytosolic pyruvate kinase gene, Acc. no STCPKIN1 (71% homologous over 58 bp),
   d) genomic sequence downstream of a Brassica napus 1.7S seed storage protein napin (napA), Acc. no. BNNAPA (71% homologous over 58 bp), and

e) 3′ untranslated region of a tomato chorismate synthase 2 precursor (CSP) gene, Acc no. LECHOSYNB (95% homologous over about 23 bp).

| a) | | | |
|---|---|---|---|
| PMGS-UQ14 | 189 | GAATCGGATATGGGTACAATAATATTTTTGAAGAGTCTG | 227 |
| | | \|\|\|\| \|\|\|\| \|\|\|\| \| \|\| \| \|\|\|\|\|\|\|\|\|\|\|\| \| | |
| Potato susi | 7710 | GAATTTGATACGGGTGCGGCAACAATTTTGAAGAGTCAG | 7748 |
| b) | | | |
| PHGS-UQ14 | 238 | TCTATGTTGCTCAGACTCTTCAAAAATATTATTGTACCCATATCCGAT | 191 |
| | | \|\|\|\|\|\|\|\|\|\|\|\|\| \| \|\|\|\|\|\|\|\|\|\| \| \|\|\|\| \| \| \| \| \|\|\| | |
| Potato LDH | 703 | TCTATGTTGCTCAAATCCTTCAAAAATGTCATTGGATGCGTGTTGGAT | 750 |
| c) | | | |
| PMGS-UQ14 | 179 | TTTTTTAAAGGAATCGGATATGGGTACAATAATATTTTTGAAGAGTCTGAGCAACATAG | 237 |
| | | \|\| \|\|\| \| \|\|\| \|\|\|\| \| \|\|\|\| \| \|\| \| \|\|\|\|\| \|\|\|\| \|\|\|\|\|\|\|\|\|\|\| | |
| Potato CPX | 951 | TTCTTTTTGAGGATCCGATACGAGTACGACAACAATTTTGGGGAGTTCGAGCAACATAG | 1009 |
| d) | | | |
| PMGS-UQ14 | 227 | CAGACTCTTCAAAAATATTATTGTACCCATATCCGATTCCTTTAAAAAAGAAAAGCTAA | 169 |
| | | \|\|\| \|\| \| \|\|\|\|\|\| \|\|\| \|\|\| \| \|\| \| \|\|\| \|\| \|\|\|\|\|\|\|\|\|\|\|\| \|\|\| | |
| napA | 2902 | CAGTCTGTACAAAAAAATTTTTGAATAAATTTAACATTATTTCAAAAAAGAAAAGGTAA | 2960 |
| e) | | | |
| PMGS-UQ14 | 202 | acaataatatttttgaagagtct | 224 |
| | | \|\|\|\| \|\|\|\|\|\|\|\|\|\|\|\|\|\|\|\|\|\| | |
| Tomato CSP | 1630 | acaacaatatttttgaagagtct | 1652 |

### EXAMPLE 8

### Tagging Additional Genes Involved in Carbon Metabolism

As the above indicates, selecting for transposition of a methylated Ds from the Dem locus and for expression of the nos:BAR gene (i.e.: demethylation of the Ds) efficiently identifies Ds insertions into regions homologous to DNA sequences of known function, as opposed to so-called “junk DNA”. In all of the above cases, the Ds insertion is in the vicinity of a region homologous to DNA sequence of known function.

The five lines carrying active nos:BAR genes associated with regions homologous to DNA sequences of known function are:
Ds insertion in UQ406-associated with the promoter of an α-amylase gene (Example 3, above);
Ds insertion in UQ12-associated with a putative patatin gene (Example 5);
Ds insertion in UQ11-associated with the Right Border of the Agrobacterium T-DNA 1516E (refer to FIGS. 12 and 13 and Example 6). This was the T-DNA carrying the Ds that was initially transformed into tomato. In other words, the Ds transposed from the Dem locus back into the T-DNA;
Ds insertion in UQ14-associated with or closely linked to a putative sucrose synthase gene (see Example 7); and
Ds insertion in UQ13-associated with or closely linked to a putative UDP-glucose-pyrophosphorylase gene and/or expansin, genes potentially involved in starch biosynthesis.

In four of these instances, the Ds is associated with DNA sequences related to carbon (C) metabolism (α-amylase, patatin, sucrose synthase and UDP-glucose-pyrophosphorylase). Since several of these lines are characterised by a disease mimic phenotype, this implies that a patatin gene and a sucrose synthase gene (and probably other C metabolism genes) are involved in disease resistance. These data also indicate that many metabolism genes and many so called house-keeping genes contain demethylation sequences or sequences which prevent or reduce methylation.

The portions of the nucleotide sequence downstream of the nos:BAR insertion in UQ13 were compared with the nucleotide sequences for tomato expansin 2 ADP-glucose pyrophosphorylase and Ca²⁺ ATPase. The Bestfit analysis is shown below:

Bestfit of UQ13D73 and Expansin 2

. . . . .

| | | |
|---|---|---|
| 510 | GGTCGTTTGGCATAAAAATACATAATGCAGGGATTATTAACGTATAGATT | 559 |
| | \| \|\|\|\| \|\| \| \|\|\| \|\| \|\|\| \| \|\|\|\| \|\| \| | |
| 4233 | GATCGTACGGTACAAAGATCAATACTTCAGG...............GAGT | 4267 |

. . . . .

| | | |
|---|---|---|
| 560 | AGTAATACATAGATTAGTAATGCATGGATTAGTTTTTATCAAGTGTTTGA | 609 |
| | \|\|\|\|\|\|\|\|\|\| \|\| \|\|\|\|\|\|\|\| \|\|\|\|\| \|\|\|\|\|\|\|\|\|\|\|\|\|\|\|\|\| | |
| 4268 | AGTAATACATTTTTTGGTAATGCAGAGATTA.TTTTTATCAAGTGTTTGG | 4316 |

. . . . .

| | | |
|---|---|---|
| 610 | TTCATTGTTTCCTACTTAATCTTATGTTTAGTTTAAAACTCTAGAAAAAT | 659 |
| | \|\|\|\|\|\|\|\|\|\| \|\|\| \|\|\|\| \|\| \|\|\| \| \|\|\|\|\|\|\| \| \|\|\|\|\|\| | |
| 4317 | TTCATTGTTT.TTACCTAATTTTGTGTGTGGTTTAAAGTTTACAAAAAAT | 4365 |

. . . . .

| | | |
|---|---|---|
| 660 | A..TATTTCCTATTATACCTTTGAGTTATTGTGAGAATTTGTATTTCATT | 707 |
| | \| \| \|\|\|\|\| \|\|\|\|\|\|\| \| \|\|\|\|\|\|\| \|\|\|\|\| \|\|\| \|\|\|\|\|\|\|\| | |
| 4366 | AATTCTTTCCAATTATACGCTAAAGTTATTATGAGATTTTATATTTCATG | 4415 |

. . . . .

| | | |
|---|---|---|
| 708 | TAACT.AGTCAAGTTAAATNCNAATTTATATATATATATATATATTATTA | 756 |
| | \|\|\| \| \|\|\|\|\| \|\| : :\|\|\|\| \| \|\|\| \|\|\| \| \|\|\|\| \| | |
| 4416 | TAATTGGGTCAA...AATAGATAATTGACCGATAATATTATTTTTTATAA | 4462 |
| 757 | ATTTT | 761 |
| | \|\|\| | |
| 4463 | CATTT | 4467 |

Bestfit UQ13D73 and Tomato ADP-glucose pyrophosphorylase

| | | |
|---|---|---|
| 542 | ATTATTAACGTATAGATTAGTAATACATAGATTAGTAATGCATGGATTAG | 591 |
| | \|\|\|\|\|\| \| \|\|\|\|\|\| \|\|\|\| \|\|\| \|\| \|\| \|\|\|\|\| \|\| \|\|\| | |
| 2035 | ATTATTGGTATCGAGATTAATAATGCATTGACTAATAATGTCGGGTTTAT | 2084 |

. .

| | | |
|---|---|---|
| 592 | TTTTTATCAAGTGTTTGATTCATT | 615 |
| | \|\|\|\|\|\|\|\|\|\|\|\|\| \|\|\|\|\| \| \| | |
| 2085 | TTTTTATCAAGTGAATGATTGAGT | 2108 |

### EXAMPLE 9

### A Rapid Bioassay for Identification of Tomato DNA Sequences Capable of Alleviating Transgene Silencing in a Heterologous Plant Species

An efficient transformation system has been developed for sugarcane, based on particle bombardment of embryogenic alleles, followed by plant regeneration (17). The bioassay is useful for identifying tomato sequences which prevent transgene silencing and employs the plant expression vector pZorz. This plasmid carries a firefly luciferase reporter gene (luc), under the control of the Osa promoter (12). After bombardment of embyrogenic callus of sugar cane, the luciferase gene is expressed, as determined by protein assay or observed by visualisation of the chemiluminescence of the luciferase enzyme. However, in normal sugarcane, it becomes completely silenced upon regeneration. The silencing appears to be correlated with methylation of the transgene. This phenomenon was used to test the effect of putative PMGSs, as follows.

Expression vector pZorz (12) was digested with HindIII and an approximately 20 bp oligonucleotide, containing a NotI restriction site and overhanging ends complementary to the HindIII site, was ligated into the HindIII site at position 1 of the pZorz backbone just upstream of the Osa promoter. The ligation results in the loss of the HindIII site. The new plasmid was designated pUQ511 (FIG. 7).

Plasmid pUQ511 was then partially digested with EcoRI, to isolate the full-length linearised plasmid. This plasmid was ligated with another approximately 20 bp oligonucleotide, containing a SmaI restriction site and overhanging ends complementary to the EcoRI site. This ligation results in the loss of the EcoRI site. Religated plasmids containing the new SmaI site at position 1370 of the pZorz backbone, just downstream of the nos terminator, were selected by PCR and this new plasmid was designated pUQ505.

Plasmid pUQ505 or pUQ511 were used as the starting vectors for constructing expression vectors containing putative PMGSs for bioassay. Tomato sequences flanking the reactivated nos:BAR insertions of UQ406, UQ11 and UQ14 were inserted into pUQ505 at the NotI site and into pUQ511 at either the NotI site or the EcoRI site or both. For example, pUQ505 was partially digested with NotI and the putative 896 bp-PMGS from UQ11, as shown in <400>9, was ligated into the new NotI site (formed as described above), in both orientations, to generate pUQ527 and pUQ5211 (FIG. 7).

These modified pZorz expression vectors were used with a transformation marker to transform sugarcane, in order to test whether the PMSGs are capable of alleviating silencing of the luc gene. Smaller fragments are then generated by deletion analysis and subcloned into expression vectors, to more accurately define the effective sequences.

Tomato sequences flanking reactivated nos:BAR in UQ406, UQ11, UQ12, UQ13 and UQ14 are also introduced next to a nos:BAR, nos:LUC or nos:GUS recombinant gene in another plasmid vector. These modified recombinant BAR, LUC and GUS genes are inserted into binary vectors (4) for transformation into Arabidopsis thaliana (18) to test the ability to prevent silencing of the nos:BAR gene in Arabidopsis.

### EXAMPLE 10

### Analysis of Sequences Responsible for Reactivating nos:BAR Expression

The borders of DNA elements that prevent transgene silencing are initially defined by deletion analysis of clones that yield positive results in the bioassays. The smallest active clone for each chromosomal region is then sequenced and characterised in detail. Sequences from independent Ds insertions are compared for homologous DNA elements.

### EXAMPLE 11

Modification of Plant Photosynthetic Architecture by Ds Transposon Tagging

As stated in Example 2; UQ406 carries a single transposed Ds element (without the transposase gene which has segregated away) and is characterised by showing an improved seedling growth, and a disease mimic or premature senescence phenotype on mature leaves. UQ406 also possesses an active nos:BAR gene indicating that the insertion caused two phenotypes: namely premature senescence and reactivation of the nos:BAR gene inside the Ds element.

Surprisingly, DNA sequence analysis shows that the Ds insertion in UQ406 is located only about 3 kb upstream from the ATG of the Dem (Defective embryo and meristems) gene which has been cloned by tagging with Ds (Example 4). In fact, only about 700 bp of DNA separates the putative α-amylase STOP codon and the Dem ATG codon (FIG. 8). This region presumably contains the promoter of Dem locus and its nucleotide sequence is shown in <400>8. The Dem gene is required for correct patterning in all of the major sites of differentiation, namely in the embryo, meristems, and organ primordia. The function of Dem was determined by STD, somatic tagging of Dem FIG. 8 provides a diagrammatic representation of the STD genotype. Mutant dem+7 is a stable frameshift mutant of Dem, TPase represents a T-DNA 3 centiMorgans (cM) from Dem, carrying the Ac transposase and a GUS reporter gene. The transposase is required for Ds transposition. The location of stably inherited (shaded) and somatic (open) Ds insertions in the Dem locus and an upstream α-amylase gene is shown in FIG. 8b. The α-amylase gene is in the same orientation as Dem Coding sequences plus introns are shown as boxes and the dark section of the Dem locus represents an intron. All of the 8 somatic insertions shown were associated with palisade deficient sectors. The genomic region represented in FIG. 8b has been sequenced (see FIG. 5; please note that the intron in the Dem locus is not included in his sequence). As shown in FIG. 8c mutant dem sectors lack palisade cells (p, palisade cells, s, spongy mesophyll, g, wild-type dark green sectors, and lg, mutant light green sectors). The inventors have shown, therefore, by somatically tagging Dem with Ds, that the gene is involved in cell growth during plant differentiation (FIGS. 8 and 9).

As stated above, the sequence flanking the active nos:BAR genes are referred to herein as “Phenotype modulating genetic sequences” or “PMGSs”.

Another genotype has been produced for the somatic tagging of the Dem gene, further demonstrating the involvement of the Dem gene in cell growth. The genetic derivation of somatically-tagged Dem is shown in FIG. 10. Besides palisade-less sectors (FIG. 8), a new phenotypic class is characterized by multicellular palisade tissue. In the wild-type tomato, the palisade tissue is composed of a single long columnar palisade cell. In the new mutant sectors, which look wild-type to the naked eye, the long columnar cell is replaced by several smaller cells packed on top of one another. This is shown in FIG. 11. Each mutant sector arises from an independent insertion of Ds in the Dem gene. The different classes of mutant sectors apparently result from different classes of mutations in the Dem gene and also indicates that Dem is involved in cell division as well as cell growth, expansion and/or division.

Somatically-tagged Dem plants are crossed to a stable null mutant of Dem and progeny are screened to identify stable mutant lines with genetically-modified palisade tissue. Lines exhibiting beneficial characteristics, such as increased levels of photosynthetic activity, can then be selected. Lines resulting from other Dem alleles and exhibiting other beneficial modifications, for example altered developmental architecture such as modified cell, tissue or organ growth rate, shape or form, may also be identified.

### EXAMPLE 12

### Transposon Tagging of α-amylase Gene

The inventors have used the transposon tagging system described in Example 4 to introduce a transposon into the α-amylase gene. One mutant line obtained was UQ406.

The DNA from 651 bp of the upstream of the UQ406 insertion down to the end of the Dem coding sequence has been sequenced (FIG. 5). The close proximity of the α-amylase gene to the Dem cell growth gene indicates that these genes may play a key role in cell growth, expansion and/or division and differentiation. Several heterozygous insertion mutants are identified in the α-amylase coding sequence and these are selfed to produce plants homozygous for the Ds insertion in the α-amylase coding sequence. If these have a similar or more or less severe phenotype to the plants homozygous for the stable Dem insertion mutant, then this will indicate that indeed this cloned α-amylase gene plays a key role in cell growth, expansion and/or division and, therefore, the shape and growth of plants.

A tomato chromosomal region spanning these genes is cloned into an Agrobacterium binary vector (19) to produce plasmid pUQ113, and this plasmid is introduced into Arabidopsis by method of Bechtold and Bouchez (18) to modify the cell shape and growth of this other plant species. A T-DNA insertion mutant in the Dem gene is identified in Arabidopsis and this mutant is also transformed with pUQ113 to modify the cell shape and growth of Arabidopsis.

Recombinant combinations of α-amylase and/or Dem genes are transformed into a range of plant species to modify the cell shape and growth of the species.

### EXAMPLE 13

### Genetic Engineering of Disease Resistance and Senescence Based on Modification of Expression of α-amylase

Ds insertion mutant UQ406 is characterize by a lesion mimic phenotype. The mutant phenotype is evident in mature leaves, but not in young leaves or any other tissue. No pathogens are found in leaf tissue displaying this phenotype. The dominant nature of the UQ406 phenotype and the location of the Ds in the α-amylase promoter suggest that over-, under or constitutive expression of the gene may be responsible for activating a disease resistance response and/or senescence in mature leaves. These data and the very close proximity of the α-amylase and Dem genes are also consistent with co-ordinate regulation of these genes in differentiating tissue. Induction of disease resistance and plant senescence, to produce desirable outcomes in crops and plant products, may, therefore, be able to be controlled by modification of α-amylase expression.

An early event in the disease response of a challenged plant is a major respiratory burst, often referred to as an oxidative burst due to an increase in oxygen consumption. This burst of oxygen consumption is due to the production of hydrogen peroxide (H₂O₂) linked to a surge in hexose monophosphate shunt activity (20). This activity results from the activation of a membrane-bound NADPH oxidase system which catalyses the single electron reduction of oxygen to form superoxide (HO₂/O₂), using NADPH as the reductant (20). Spontaneous dismutation of HO₂/O₂⁻then yields H ₂O₂. Consumption of glucose via the hexose monophosphate shunt (alternatively known as the cytosolic oxidative pentose phosphate pathway) regenerates the NADPH consumed by the NADPH oxidase system. It is, therefore, entirely conceivable that an α-amylase is responsible for supplying sugars required by the pentose phosphate pathway, and perhaps for the primary activation of the signal transduction pathway that leads to disease resistance in plants.

Following the oxidative burst, disease resistance is manifested in localised plant cell death called the hypersensitive response (HR), in the vicinity of the pathogen. The HR may then induce a form of long-lasting, broad spectrum, systemic and commercially important resistance known as systemic acquired resistance (SAR). The compounds, salicylic acid, jasmonic acid and their methyl derivatives as well as a group of proteins known as pathogenesis related (PR) proteins are used as indicators of the induction of SAR (23).

Increased levels of sugars have been r elated to heightened resistance especially to biotrophic pathogens (21). When invertase (the enzyme responsible for the breakdown of sucrose to glucose and fructose) is overexpressed in transgenic tobacco, systemic acquired resistance is induced (22).

The α-amylase coding sequence is inserted behind an inducible promoter and transformed into plants to confer a inducible disease resistance in plants. Similarly, the α-amylase coding sequence is inserted behind an inducible promoter and transformed into plants to confer inducible senescence in plants for the production of desirable products or traits.

When a disease resistance response is invoked in one part of a plant, a general and systemic acquired enhancement in disease resistance is conferred on all tissues of such a plant (21). Tomato line UQ406 is tested for enhanced resistance to a wide range of pathogens to test this hypothesis.

### EXAMPLE 14

### Modifications of Carbon Metabolism

As stated in Examples 7 and 8, in four of the five lines carrying active demethylated nos:BAR genes, the Ds has inserted into or near sequences homologous with carbon metabolism gene. These results indicated that many C metabolism genes have cis-acting sequences which prevent methylation and concomitant gene silencing. Demethylation sequences are inserted next to recombinant C metabolism genes to enhance their expression and modify C metabolism in beneficial ways; such as up-regulation of the sucrose phosphate synthase gene in sugar cane, to yield higher concentrations of sugar in beneficially-modified plants.

### EXAMPLE 15

### Cloning of Downstream Genes Associated with Plant Cell Apoptosis Caused by Ds Insertion

A cDNA library is made from tomato leaf tissue showing the disease mimic (apoptosis) phenotype caused by Ds insertion in UQ406. This library is screened differentially with two probes, one being cDNA from normal tissue and the other being cDNA made from leaf tissue showing the disease mimic phenotype caused by Ds insertion. This procedures identifies genes specifically-induced during plant cell death. These apoptosis-associated genes are then sequenced, and compared with other genes present in the DNA databases. The proteins encoded by these genes are expressed in vitro and tested for their ability to kill plant cells.

### EXAMPLE 16

### Analysis of Dem and its Product DEM

1. DEM in Differentiating Cells

A truncated version of DEM protein is expressed in vitro from an E. coli pET expression vector. Polyclonal antibody is raised against this truncated DEM protein in mice. In Western blots, the polyclonal antibody specifically recognizes a protein of the predicted size of the DEM protein in shoot meristem tissue. This antibody is employed in immunolocalization experiments. Tomato shoot and root meristematic regions and leaf primordia are processed for electron microscopy and immunolocalization of DEM. The technique employs gentle aldehyde crosslinking of the tissues and infusion with saturated buffered sucrose before freezing the samples in liquid nitrogen. Mounted blocks are then thin sectioned at low temperature at low temperature and immunolabelled with fluorescent or electron dense markers suitable for electron microscopy, a room temperature. An advantage of this methodology is the excellent ultrastructural preservation, combined with the retention of antigenicity which allow for meaningful antigen-antibody localisation of proteins. Results show that the polyclonal antibody detects an antigen in the outer cell layer of shoot meristem tissue.

2. Cell Walls

Stand analytical techniques are used to analyse and compare cell wall compositions of mutant dem and wild-type tissue.

3. Function of the DEM Homologue (YNV212N) in Yeast

The mature N-terminal sequence of the DEM protein, MGANHS conforms to the consensus sequence for N-myristoylation. This consensus sequence appears to be missing from the predicted YNV212W protein based on genomic sequence. A full length yeast YNV212W cDNA is cloned and sequenced, and gene disruption techniques are used to introduce frameshift mutations at several locations along the YNV212W coding sequence. By generating frameshift mutations at several points along the gene, mutant alleles of YNV212W are created. The resultant mutants are observed for modified growth and morphology. There are no other genes in yeast that are homologous to YNV212W. YNV212W cDNA is cloned into an inducible expression vector for yeast, and yeast strains overexpressing YNV212W are observed for changes in growth and morphology.

4. Identification of Wild-Type and Mutated Arabidopsis Genes that are Homologous to Dem, and Observation of Insertion Mutants for Altered Morphology

BLAST searches (25) using the tomato Dem nucleotide sequence has identified three separate homologous sequences in Arabidopsis (accession numbers AB020746, AC000103 and ATTS5958). The level of homology to the tomato gene ranges from 56 to 68% on the nucleotide level over 350 to 800 bp and indicates that there may be several genes related to Dem in plants. Full length Arabidopsis cDNAs homologous to the tomato Dem cDNA are cloned and sequenced. Antisense constructs under control of te cauliflower mosaic virus 35S promoter are made and transformed into Arabidopsis and the resulting transformants are observed for morphological abnormalities. Insertion mutants in Dem homologues are identified from the dSpm and T-DNA tagged lines of Arabidopsis. Insertion mutants are screened for modified morphology.

5. Identification and Characterization of Additional Stable Ds Insertions in the Vicinity of Dem and Screening for Mutants with Modified Photosynthetic Architecture

Up to 2,000 STD progeny lacking the Ac transposase (detected by absence of the GUS reporter gene) are screened by PCR for Ds insertions in the region of Dem DNA is extracted from bulk leaf samples of 50 plants and used as template in 8 PCRs. All 8 reactions include oligonucleotide primers facing away from both sides of Ds. The 8 separate PCRs vary according to the oligonucleotide primer used to anneal to the tomato genomic sequence. These 8 primers are evenly distributed, 1 kb apart along the tomato sequence. Amplification of a fragment indicates a Ds insertion in the vicinity of Dem When a fragment is amplified from a DNA sample, the PCR product is authenticated by a nested PCR. Subsequently, the individual plant carrying the Ds insertion in the vicinity of Dem is identified by the appropriate PCR assay, using intact leaf tissue as template. Plants homozygous for new stable Ds insertions in the vicinity of the Dem locus are morphologically characterized, both in terms of meristem structure and organization of photosynthetic tissue. New lines showing modified morphology are crossed to a line expressing Ac transposase. Instability of the phenotype in the presence of transposase will confirm that a Ds element is responsible for the modified morphology.

The progeny from STD plants are also screened directly for stable mutants in the photosynthetic architecture of leaves. The screen involves hand-sectioning the tissue, then toluidine blue staining followed by light microscopy. This method results in the isolation of genetically-stable multicellular palisade mutants. Mutants are crossed to a line expressing Ac transposase to determine if the mutation is due to a Ds insertion. If the phenotype shows instability in the presence of transposase, the corresponding gene is cloned and characterized.

6. Antisense Dem Constructs for Transformation into Tomato

Antisense constructs involving the tomato Dem coding sequence are produced and transformed into tomato with the aim of producing a large number of tomato lines that vary in DEM function. The antisense constructs are made under the control of the 35S promoter. Thirty transformants are produced and observed for modified growth and morphology. Microscopy is used to characterize the organization of photosynthetic tissue in these antisense lines.

### EXAMPLE 17

### Analysis of PMGSs

The PMGSs in mutant lines such as UQ11, 12, 13 and 14 and 406 are analysed in a number of ways. In one analysis, the right border (RB) and or flanking DNA in a Ds containing line in which nos:BAR is expressed is used to screen for stabilize expression of transgenes. For convenience, transgenes encode a reporter molecule capable of providing an identifiable signal. Examples of such reporter transgenes include antibiotic resistance.

In addition, genetic constructs comprising nucleotide sequences carrying PMGSs flanking nos:BAR are inserted next or otherwise proximal to selectable transformation marker genes such as BAR or PT and the resulting plasmids are used in transformation experiments to enhance the transformation efficiency of plant species such as wheat and sugar cane.

### EXAMPLE 18

### Therapeutic Application of PMGSs

Latent viruses such as HIV-1 may employ mechanisms such as methylation to remain inactive until de-methylation occurs. The PMGSs of the present invention may be used to de-methylate and activate latent viruses such as HIV-1 so that such viruses can then be destroyed or inactivated by chemical or biological therapeutic agents.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

1 McClintock, B. (1947) Carnegie Inst. Washington Year Book 46: 146-152.
2 McClintock, B. (1948) Carnegie Inst. Washington Year Book 47: 155-169.
3 Fedoroff, N. et at, (1984) Proc. Natl. Acad. Sci USA 81: 3825-3829.
4 Jones, J. et al. (1992) Transgenic Res. 1: 285-297.
5 Carroll, B. J. et al. (1995) Genetics 139: 407-420.
6 Scofield, S. et al. (1992) Plant Cell 4: 573-582.
7 Finnegan, J and McElroy, D (1994) Biotech 12: 883-888.
8 Spiker, S and Thompson, W F (1996) Plant Physiol 110: 15-21.
9 Matzke, M A and Matzke, A J M Plant Physiol 107: 679-685.
10 Jorgensen, R A (1995) Science 268: 686-691.
11 Smith, H A et al (1994) Plant Cell 6: 1441-1453.
12 Bower, R et al (1996) Mol. Breeding 2: 239-249.
13 Keddie, J S et al (1996) EMBO J. 15: 4208-4217.
14 Siebert, P. D. et at. (1995) Nucleic Acids Res. 23: 1087-1088.
15 International Patent Publication No. WO 96/12813.
16 International Patent Publication No. WO 90/12876.
17 Bower, R and Birch, R G (1992) Plant J. 2: 409-416.
18 Bechtold, N. and Bouchez, D. (1995) In: I. Potrykus and G. Spangenberg (eds). Gene transfer to Plants. pp. 19-23.
19 Dixon, M. S. et al. (1996) Cell 84: 451-459.
20 Pugin, A. et al. (1997) Plant Cell 9: 2077-2091.
21 Vanderplank, I. E. (1984) “Sink-induced loss of resistance”. In Disease resistance in plants (2nd Ed.), J. E. Vanderplank, ed. (London: Academic Press), pp. 107-116.
22 Herbers, K., Meuwly, P., Frommer, W. B., Metraux, J-P., and Sonnewald, U. (1996). The Plant Cell 8: 793-803.
23 Ryals, J. et al (1995) Proceedings of the National Academy of Science USA 92: 4202-4205.
24 Needleman and Wunsch (1970) J. Mol. Biol. 48: 443-453.
25 Altschul S F, Gish W, Miller W, Myers E W, Lipman D (1990) J Mol Biol 215: 403-410.

1. (canceled)

2. The PMGS according to claim 27 wherein said PMGS promotes demethylation or prevents or inhibits methylation of a second nucleotide sequence located within, adjacent to, or otherwise proximal to said PMGS.

3-6. (canceled)

7. A genetic construct comprising the PMGS according to claim 27 and means to facilitate insertion of a nucleotide sequence within, adjacent to, or otherwise proximal to said PMGS.

8. The genetic construct according to claim 29 wherein the inserted nucleotide sequence is operably linked to a promoter.

9. A method of increasing or stabilizing expression of a nucleotide sequence or preventing or reducing silencing of a nucleotide sequence in a plant, comprising: introducing said nucleotide sequence into the plant, such that when expressed in the plant, said nucleotide sequence is flanked by, adjacent to, or otherwise proximal to, the PMGS of claim 27.

10-19. (canceled)

20. An isolated nucleotide sequence having at least 80% similarity after optimal alignment of said sequence to the PMGS of claim 27, wherein said PMGS Increases or stabilizes the expression of a second nucleotide sequence inserted proximal to said PMGS.

21-22. (canceled)

23. A sequence capable of hybridizing to the PMGS according to claim 27 under medium stringency conditions at 42° C. comprising from at least about 16% v/v to at least about 30% v/v formamide and from at least about 0.5M to at least about 0.9M salt for hybridization and at least about 0.5M to at least about 0.9M salt for washing.

24. A sequence capable of hybridizing to the PMGS according to claim 27 under high stringency conditions at 42° C. comprising from at least about 31% v/v to at least about 50% v/v formamide and from at least about 0.01 M to at least about 0.15M salt for hybridization and at least about 0.01M to at least about 0.15M salt for washing.

25. A method of increasing or stabilizing expression of a nucleotide sequence, or preventing or reducing silencing of a nucleotide sequence, in a plant or plant cell, comprising:
introducing into said plant or plant cell a construct comprising said nucleotide sequence whose expression is to be increased or stabilized, or whose silencing is to be prevented or reduced, flanked by, adjacent to, or otherwise proximal to the PMGS of SEQ ID NO:12 and SEQ ID NO. 13; and
expressing said construct in said plant or plant cell.

26. The method according to claim 9 or claim 25, wherein the prevention or reduction in silencing of said nucleotide sequence is by prevention of methylation, or enhancement of demethylation of said nucleotide sequence.

27. An isolated phenotype modulating genetic sequence (PMGS) comprising the nucleotide sequences of SEQ ID NO:12 and SEQ ID NO:13.

28. The genetic construct of claim 7, wherein said means to facilitate insertion is between the nucleotide sequence of SEQ ID NO:12 and the nucleotide sequence of SEQ ID NO:13.

29. The genetic construct of claim 28 which further comprises an inserted nucleotide sequence.

30. The genetic construct of claim 8, wherein said inserted nucleotide sequence promotes transcript degradation.

31. An Isolated nucleic acid comprising the nucleotide sequence of SEQ ID NO:12, SEQ ID NO:13 or both.

32. A genetic construct comprising the Isolated nucleic acid according to claim 31 and means to facilitate insertion of a first nucleotide sequence within, adjacent to, or otherwise proximal to the nucleotide sequence of SEQ ID NO:12, SEQ ID NO:13 or both, if present.

33. The genetic construct of claim 32 wherein said means to facilitate insertion is within the PMGS.

34. The genetic construct of claim 33 which further comprises an inserted nucleotide sequence.

35. The genetic construct according to claim 34 wherein the nucleotide sequence to be inserted is operably linked to a promoter.

36. The genetic construct of claim 35, wherein said inserted nucleotide sequence promotes transcript degradation.

37. The method according to claim 9 or claim 25, wherein said nucleotide sequence promotes transcript degradation.

38. The method according to claim 9 or claim 25, wherein said nucleotide sequence is flanked by SEQ ID NO. 12 on one side and SEQ ID NO. 13 on the other side.

39. A method for inducing or increasing pathogen resistance in a plant which comprises introducing into said plant a genetic construct comprising a PMGS flanked by, adjacent to, or otherwise proximal to a nucleotide sequence that promotes transcript degradation or co-suppression of plant virus, wherein said PMGS comprises SEQ ID NO:12 and SEQ ID NO:13.

40. The method according to claim 39, wherein said nucleotide sequence is flanked by SEQ ID NO. 12 on one side and SEQ ID NO. 13 on the other side.
